(19) United States
(12) Patent Application Publication
(10) Pub. No.: US 2022/0009910 A1
(43) Pub. Date: Jan. 13, 2022

(54) TYROSINE KINASE 2 INHIBITORS, PREPARATION METHODS AND MEDICINAL USES THEREOF

(71) Applicant: Eternity Bioscience Inc., Cranbury, NJ (US)

(72) Inventors: Linghang Zhuang, Chalfont, PA (US); Jian Liu, Manalapan, NJ (US); Yinfa Yan, Bedminster, NJ (US); Puhui Li, Plainsboro, NJ (US); Yu Zhou, East Brunswick, NJ (US); Suxing Liu, Edison, NJ (US)

(21) Appl. No.: 17/372,454

(22) Filed: Jul. 10, 2021

Related U.S. Application Data
(60) Provisional application No. 63/050,716, filed on Jul. 10, 2020.

(51) Int. Cl. C07D 403/14; C07D 405/14

(52) U.S. Cl. CPC C07D 403/14; C07D 405/14

(57) ABSTRACT

This application discloses Tyk-2 inhibitors represented by the general formula (I) and analogs thereof, pharmaceutical compositions containing these compounds, method of preparing them, and use of these compounds as therapeutic agents for the treatment of diseases or conditions associated with Tyk-2 activity, such as autoimmune diseases and cancers.

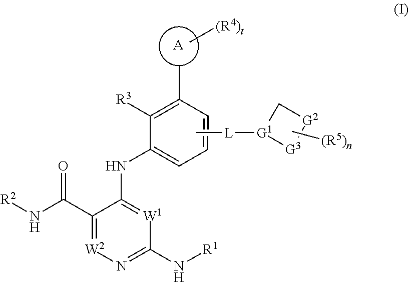

### CROSS-REFERENCE TO RELATED APPLICATIONS

This application claims priority under 35 U.S.C. § 119(e) to U.S. Provisional Patent Application No. 63/050,716, filed on Jul. 10, 2020, the disclosure of which is incorporated herein by reference in its entirety.

### FIELD OF THE DISCLOSURE

The present disclosure relates to compounds useful in the suppression of non-receptor tyrosine-protein kinase 2, also known as Tyrosine kinase 2 (Tyk2). The disclosure further provides pharmaceutical compositions containing these compounds and methods of using the pharmaceutical compositions in the treatment of various disorders related to the regulation of Tyk2 activity.

### BACKGROUND OF THE DISCLOSURE

Cytokine signaling plays a pivotal role in controlling the growth, differentiation, function, and communication of immune cells. Multiple cytokine signaling transduction pathways are mediated by the actions of receptor-bound Janus kinases (JAKs) and the signal transducers and activators of transcription (STATs) (Cooper, G S et al., J. Autoimmun. 2009, 33: 197-207; Schwartz D M et al., Nat Rev Drug Discov. 2017, 17:78; Schwartz D M et al., Nat Rev Rheumatol. 2016, 12: 25-36).

The JAKs are a family of non-receptor tyrosine kinases (JAK1, JAK2, JAK3, and TYK2) that associate with the intracellular domains of cell surface cytokine receptors. Upon stimulation and oligomerization of these receptors, the JAK molecules are activated and serve as docking sites for subsequent recruitment and phosphorylation of STAT proteins. In turn, the phosphorylated STAT proteins then dimerize, translocate to the nucleus, and activate transcription of genes mediating cytokine-induced responses. These cytokine-mediated-JAK/STAT pathways are tightly regulated, and dysfunctional JAK/STAT activities have been demonstrated as hallmarks of numerous immunological and autoimmune disorders, inflammatory diseases, as well as cell transformation (Schwartz D M et al., Nat Rev Drug Discov. 2017, 17:78).

Tyrosine kinase 2 (Tyk2), the first identified member of the JAK family, is a major component in various cytokine pathways, resulting in the STAT-dependent gene transcription and specific functional responses of the cytokines, which include the Interleukin-12/-23 family (IL-12/IL-23, which share a common p40 subunit), the Type I interferon (IFN) family, as well as the IL-6 and IL-10 families (Schwartz D M et al., Nat Rev Rheumatol. 2016, 12: 25-36). The Tyk2-mediated signaling of cytokines play critical roles in autoimmune disorders and inflammatory disease pathogenesis. Specifically, IL-23 (a heterodimer that contains p40 and p19 subunits) is crucial for the differentiation and proliferation of T helper cells 17 (Th17), which are a key participant in several autoimmune diseases (Aggarwal, S et al., J Biol Chem. 2003, 278: 1910-1914). IL-12, which is composed of p40 and a unique p35 subunit, is important in regulating Th1 development and the IFN-γ secretion of these cells (Metzger D W at al., Eur J Immunol. 1997, 27:1958-65). Through the mediation of Th1/Th17 responses, IL-12 and IL-23 play essential roles in a variety of inflammatory diseases, such as Psoriasis (Ps), Lupus, Inflammatory Bowel Disease (IBD), Multiple Sclerosis (MS), Rheumatoid Arthritis (RA), etc (Michele W L T et al., Nat Med. 2015, 21:719-729; Andrew L. C et al., Eur. J. Immunol. 2012, 42: 2263-2273; Craig A. Metal., J. Exp. Med. 2003, 198:1951-1957). For example, in mouse models, deficiency of either the common subunit P40, or shared receptor IL23R, of IL12 and IL23, were found to protect mice from various autoimmune diseases (Ps, Lupus, IBD, MS, RA, etc.) (Kyttaris V C et al., J Immunol. 2010, 184:4605-9; Paulina K et al., Nat Commun. 2016, 7: 13466; Hong K et al., J Immunol. 1999, 162:7480-91). In human disease, high levels of IL-12 and IL-23 were observed in the lesional skin of psoriatic patients, which was then lowered after various therapeutic treatments of psoriasis (Lee E at al., J Exp Med. 2004, 199:125-30). Moreover, blocking monoclonal antibodies directed against the IL-12/IL-23 common subunit p40 (Ustekinumab, Briakinumab, etc.), or IL-23 specific subunit p19 (Tildrakizumab, Risankizumab, etc), were proven to be clinically efficacious in treating psoriasis, Crohn's disease, etc (Gandhi M at al., Semin Cutan Med Surg. 2010, 29:48-52; Bram V et al., J Gastroenterol. 2018, 53: 585). Meanwhile, the Type I IFN family members (IFN-α, -β, -δ, -κ, and -ω), acting through a heterodimer IFN receptor (IFNAR), are not only important mediators for both innate and adaptive immunities by activating numerous factors in immune responses, but also effective enhancers of autoantigen expression and release, thus becoming crucial participants in autoimmune disease amplification (Lionel B. I. et al., Nat Rev Immunol. 2014, 14: 36-49; John C. H. et al., Nat Rev Rheumatol. 2010, 6: 40-49; Antonios P et al., Rheumatology 2017, 10: 1662-1675). The importance of Type I IFNs in pathogenesis of Systemic Lupus Erythematosus (SLE) is evidenced by the observation that deletion of the IFNAR in lupu-prone NZB mice provides a high degree of protection from lupus severity and mortality (Santiago-Raber M L et al., J Exp Med. 2003, 197:777-88). In human SLE patients, elevated serum IFNα levels and increased type-I IFN regulated gene expression in peripheral blood mononuclear cells (PBMCs) and affected organs were reported in majority of patients. Furthermore, several studies reported the activation of the Type I IFN genes are well-correlated with activity and the severity of SLE (Bengtsson A A et al., Lupus. 2000, 9:664-71).

Emerging research evidence demonstrated the importance of Tyk2 in the pathogenesis of afore-mentioned autoimmune diseases. For example, Tyk2-deficient or -chemically inhibited rodents were revealed to be resistant to experimental autoimmune disease models of psoriasis, MS, and IBD (Michael P. S. et al., Front. Immunol. 2018, 10: 3389; Ishizaki Metal., J Immunol. 2011, 187:181-9; Miao W et al., Arthritis Rheumatol. 2016, 68, suppl 10). In human studies, catalytically impaired Tyk2 variants (r512720356, and rs34536443) are found to be protective against childhood-, as well as adult-, onset of SLE in the Mexican Mestizo population (Cecilia C. C. et al., Scientific Reports. 2019, 9: 12165). In addition, Tyk2 SNP mutations were also shown to be associated with SLE in patients of Nordic ancestry, UK, and Han Chinese populations. More broadly, Genome-wide association studies (GWAS) have identified several inactive variants of Tyk2 to be significantly associated with inflammatory diseases, including multiple sclerosis, psoriasis, Crohn's disease, lupus, and rheumatoid arthritis, further indicating the impact of Tyk2 in a broad scope of autoimmune disorders (Westra H J et al., Nat Genet. 2018, 50:1366-1374; Okada Y et al., Nature. 2014, 506:376-81; Mero I L et al., Eur J Hum Genet. 2010, 18:502-4; Peluso C et al., Hum Immunol. 2013, 74:93-7; Gorman J A et al., Front Immunol. 2019, 25; 10:44). Therefore, it is rationalized that agents that suppress the action of Tyk2-mediated cytokine signaling pathways may have potential therapeutical benefit in human autoimmune diseases. Indeed, a highly selective allosteric Tyk2 inhibitor, BMS986165, was shown to effectively block IL-12/IL-23 and Type I IFN pathways and thus demonstrate substantial in vivo efficacy in numerous experimental autoimmune disease models (psoriasis, SLE, and IBD) (Tokarski J S et al., J Biol Chem. 2015, 290:11061-74). Moreover, the Phase 2 trial results of this agent was announced to achieve ≥75% and 90% reduction in the Psoriasis Area and Severity Index (PASI 75, PASI 90) in patients with moderate to severe plaque psoriasis with a favorable risk-benefit profile (Kim P. et al., N Engl J Med. 2018, 379:1313-1321), further supporting the notion of Tyk2 as a promising therapeutic target in the area of autoimmune-disorders.

### SUMMARY OF THE DISCLOSURE

The present disclosure, in one aspect, provides a compound of formula (I), or a tautomer, mesomer, racemate, enantiomer, diastereomer, or mixture thereof, or a pharmaceutically acceptable salt, solvate or prodrug thereof:

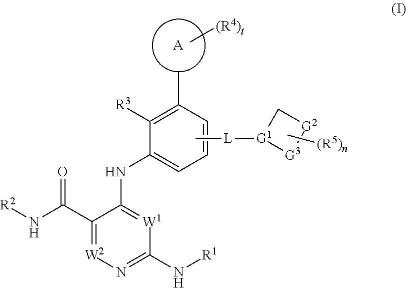

wherein:

G¹ is N atom or C atom;

G² and G³ are identical or different, and each is independently selected from the group consisting of C atom, O atom, N atom and S atom, provided that at least one of G¹, G² and G³ is a heteroatom selected from the group consisting of N, O and S;

W¹ and W² are identical or different, and each is independently N atom or CR⁶;

L is selected from the group consisting of bond, O atom and alkylene, wherein the alkylene is optionally substituted with one or more, sometimes preferably one to three, groups independently selected from the group consisting of halogen, alkyl, alkoxy, hydroxyl, hydroxyalkyl, cyano, amino, nitro, cycloalkyl, heterocyclyl, aryl and heteroaryl;

ring A is heteroaryl;

R¹ is selected from the group consisting of hydrogen, alkyl, deuterated alkyl, haloalkyl, hydroxyalkyl, alkenyl, alkynyl and —C(O)R⁷;

R² is selected from the group consisting of hydrogen, halogen, alkyl, deuterated alkyl, alkenyl, alkynyl, alkoxy, haloalkyl, haloalkoxy, hydroxyl, hydroxyalkyl, cyano, amino, nitro, cycloalkyl and heterocyclyl;

R³ is selected from the group consisting of hydrogen, halogen, alkyl, deuterated alkyl, alkenyl, alkynyl, alkoxy, haloalkyl, haloalkoxy, hydroxyl, hydroxyalkyl, cyano, amino nitro, cycloalkyl and heterocyclyl;

R⁴ is each identical or different, and each is independently selected from the group consisting of hydrogen, halogen, alkyl, deuterated alkyl, alkenyl, alkynyl, alkoxy, haloalkyl, haloalkoxy, hydroxyl, hydroxyalkyl, cyano, amino, nitro, cycloalkyl, heterocyclyl, aryl and heteroaryl;

R⁵ is each identical or different, and each is independently selected from the group consisting of hydrogen, halogen, alkyl, oxo, alkenyl, alkynyl, alkoxy, haloalkyl, haloalkoxy, hydroxyl, hydroxyalkyl, cyano, amino, nitro, cycloalkyl, heterocyclyl, aryl and heteroaryl;

R⁶ is selected from the group consisting of hydrogen, halogen, alkyl, alkenyl, alkynyl, deuterated alkyl, alkoxy, haloalkyl, haloalkoxy, hydroxyl, hydroxyalkyl, cyano, amino, nitro, cycloalkyl, heterocyclyl, aryl and heteroaryl;

R⁷ is selected from the group consisting of alkyl, alkenyl, alkynyl, haloalkyl, hydroxyalkyl, cycloalkyl and heterocyclyl;
n is 0, 1, 2, 3 or 4; and
t is 0, 1, 2 or 3.

In another aspect, the present disclosure also provides a pharmaceutical composition, comprising a therapeutically effective amount of a compound of formula (I), or a tautomer, mesomer, racemate, enantiomer, diastereomer, or mixture thereof, or a pharmaceutically acceptable salt, solvate or prodrug thereof, together with one or more pharmaceutically acceptable carriers, diluents or excipients.

In another aspect, the present disclosure provides a method of treating a Tyk2-mediated disorder, disease or condition, comprising administering to a subject in need thereof a therapeutically effective amount of a compound of formula (I), or a tautomer, mesomer, racemate, enantiomer, diastereomer, or mixture thereof, or a pharmaceutically acceptable salt, solvate or prodrug thereof, or a pharmaceutical composition containing the same.

In another aspect, the present disclosure relates to a method of treating proliferative, metabolic, allergic, autoimmune and inflammatory diseases, comprising a step of administering to a subject in need thereof a therapeutically effective amount of the compound of formula (I), or a tautomer, mesomer, racemate, enantiomer, diastereomer, or mixture thereof, or a pharmaceutically acceptable salt, solvate or prodrug thereof, or a pharmaceutical composition containing the same.

In another aspect, the present disclosure relates to a method of treating autoimmune and inflammatory diseases, comprising a step of administering to a subject in need thereof a therapeutically effective amount of the compound of formula (I), or a tautomer, mesomer, racemate, enantiomer, diastereomer, or mixture thereof, or a pharmaceutically acceptable salt, solvate, or prodrug thereof, or pharmaceutical composition containing the same. The autoimmune and inflammatory diseases is selected from arthritis, rheumatoid arthritis, multiple sclerosis, systemic lupus erythematosus, lupus nephritis, cutaneous lupus, inflammatory bowel disease, psoriasis, psoriatic arthritis, Crohn's disease, Sjögren's syndrome, systemic scleroderma, ulcerative colitis, Graves' disease, discoid lupus erythematosus, adult onset stills, systemic onset juvenile idiopathic arthritis, gout, gouty arthritis, type I diabetes, insulin dependent diabetes mellitus, sepsis, septic shock, Shigellosis, pancreatitis, glomerulonephritis, autoimmune gastritis, diabetes, autoimmune hemolytic anemia, autoimmune neutropenia, thrombocytopenia, atopic dermatitis, myasthenia gravis, ankylosing spondylitis, pemphigus vulgaris, Goodpasture's disease, antiphospholipid syndrome, idiopathic thrombocytopenia, ANCA-associated vasculitis, pemphigus, Kawasaki disease, Chronic Inflammatory Demyelinating Polyneuropathy (CIDP), dermatomyositis, polymyositis, uveitis, Guillain-Barre syndrome, autoimmune pulmonary inflammation, autoimmune thyroiditis, autoimmune inflammatory eye disease and chronic demyelinating polyneuropathy.

In another aspect, the present disclosure relates to a method of treating cancer, comprising a step of administering to a subject in need thereof a therapeutically effective amount of the compound of formula (I), or a tautomer, mesomer, racemate, enantiomer, diastereomer, or mixture thereof, or a pharmaceutically acceptable salt, solvate, or prodrug thereof, or pharmaceutical composition containing the same. wherein the cancer can be selected from the group consisting of breast cancer, cervical cancer, colon cancer, lung cancer, gastric cancer, rectal cancer, pancreatic cancer, brain cancer, skin cancer, oral cancer, prostate cancer, bone cancer, kidney cancer, ovarian cancer, bladder cancer, liver cancer, tubal tumor, ovarian tumor, peritoneal tumor, melanoma, glioma, neuroblastoma, hepatocellular carcinoma, papillomatosis, head and neck tumor, leukemia, lymphoma, myeloma and non-small cell lung cancer.

The inhibition of JAK family members may directly and indirectly suppress the activity of multiple cytokines that are involved in physiological immune functions. Thus, differences may be found between wide-ranging inhibition that suppresses the signaling of multiple physiological mediators, and selective inhibition that may spare other members of the JAK family and thereby avoid corresponding safety concerns. Data from previous studies revealed that non-selective inhibition of JAK1, 2, and 3 was associated with an increased risk of serious infections and opportunistic infections. Also, dose-dependent changes in laboratory parameters, including lipids, levels of hemoglobin, decreased numbers of lymphocytes, NK cells, neutrophils, and platelets have been observed, as well as cases of venous thromboembolism and gastrointestinal perforation (Miguel N et al., Drugs. 2020; 80: 341-352).

On the other hand, selective TYK2 inhibitors have demonstrated significant potential regarding oral treatment of moderate-to-severe psoriasis. This selective TYK2 inhibitor did not increase the incidence of herpes zoster and thromboembolic events, and the same happened with dyslipidaemia, a common effect of JAK1 inhibitors mediated through IL-6 signaling impairment (Charles-Schoeman C, et al. Arthritis Rheumatol, 2015; 67:616-25); thus, Tyk2 selective inhibitors might be considered to belong to a different therapeutic class compared to nonselective JAKi.

Taken together, many diseases may be associated with abnormal cellular responses triggered by Tyk2-mediated events. These diseases include, but are not limited to, autoimmune diseases, inflammatory diseases, cancer, graft versus host disease, metabolic diseases, allergies and asthma, cardiovascular diseases, bone diseases, neurological and neurodegenerative diseases, Alzheimer's disease, and hormone-related diseases. Accordingly, the present disclosure provides selective Tyk2 inhibitors useful as therapeutic agents.

### DETAILED DESCRIPTION OF THE DISCLOSURE

In one aspect, the present disclosure provides a compound of formula (I), or a tautomer, mesomer, racemate, enantiomer, diastereomer, or mixture thereof, or a pharmaceutically acceptable salt, solvate, or prodrug thereof:

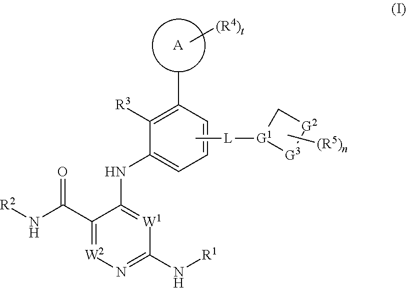

wherein:

G¹ is N atom or C atom;

G² and G³ are identical or different, and each is independently selected from the group consisting of C atom, O atom, N atom and S atom, provided that at least one of G¹, G² and G³ is a heteroatom selected from the group consisting of N, O and S;

W¹ and W² are identical or different, and each is independently N atom or CR⁶;

L is selected from the group consisting of bond, 0 atom and alkylene, wherein the alkylene is optionally substituted with one or more groups selected from the group consisting of halogen, alkyl, alkoxy, hydroxyl, hydroxyalkyl, cyano, amino, nitro, cycloalkyl, heterocyclyl, aryl and heteroaryl;

ring A is heteroaryl;

R¹ is selected from the group consisting of hydrogen, alkyl, deuterated alkyl, haloalkyl, hydroxyalkyl, alkenyl, alkynyl and —C(O)R⁷;

R² is selected from the group consisting of hydrogen, halogen, alkyl, deuterated alkyl, alkenyl, alkynyl, alkoxy, haloalkyl, haloalkoxy, hydroxyl, hydroxyalkyl, cyano, amino, nitro, cycloalkyl and heterocyclyl;

R³ is selected from the group consisting of hydrogen, halogen, alkyl, deuterated alkyl, alkenyl, alkynyl, alkoxy, haloalkyl, haloalkoxy, hydroxyl, hydroxyalkyl, cyano, amino nitro, cycloalkyl and heterocyclyl;

R⁴ is each identical or different, and each is independently selected from the group consisting of hydrogen, halogen, alkyl, deuterated alkyl, alkenyl, alkynyl, alkoxy, haloalkyl, haloalkoxy, hydroxyl, hydroxyalkyl, cyano, amino, nitro, cycloalkyl, heterocyclyl, aryl and heteroaryl;

R⁵ is each identical or different, and each is independently selected from the group consisting of hydrogen, halogen, alkyl, oxo, alkenyl, alkynyl, alkoxy, haloalkyl, haloalkoxy, hydroxyl, hydroxyalkyl, cyano, amino, nitro, cycloalkyl, heterocyclyl, aryl and heteroaryl;

R⁶ is selected from the group consisting of hydrogen, halogen, alkyl, alkenyl, alkynyl, deuterated alkyl, alkoxy, haloalkyl, haloalkoxy, hydroxyl, hydroxyalkyl, cyano, amino, nitro, cycloalkyl, heterocyclyl, aryl and heteroaryl;

R⁷ is selected from the group consisting of alkyl, alkenyl, alkynyl, haloalkyl, hydroxyalkyl, cycloalkyl and heterocyclyl;
n is 0, 1, 2, 3 or 4; and
t is 0, 1, 2 or 3.

In some embodiments of the disclosure, the compound of formula (I), or a tautomer, mesomer, racemate, enantiomer, diastereomer, or mixture thereof, or a pharmaceutically acceptable salt, solvate, or prodrug thereof, is a compound of formula (II), or a tautomer, mesomer, racemate, enantiomer, diastereomer, or mixture thereof, or a pharmaceutically acceptable salt, solvate, or prodrug thereof:

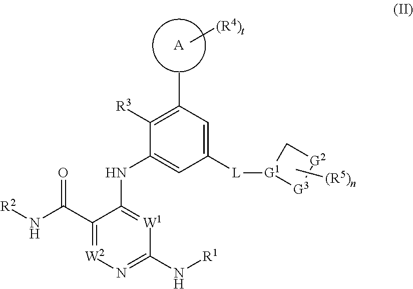

wherein:

ring A, L, G¹, G², G³, W¹, W², R¹ to R⁵, n and t are each as defined in formula (I) above.

In some embodiments of the disclosure, in the compound of formula (I), or a tautomer, mesomer, racemate, enantiomer, diastereomer, or mixture thereof, or a pharmaceutically acceptable salt, solvate, or prodrug thereof, wherein W¹ is CR⁶, and W² is N atom; R⁶ is as defined in formula (I) above.

In some embodiments of the disclosure, the compound of formula (I), or a tautomer, mesomer, racemate, enantiomer, diastereomer, or mixture thereof, or a pharmaceutically acceptable salt, solvate, or prodrug thereof, is a compound of formula (III), or a tautomer, mesomer, racemate, enantiomer, diastereomer, or mixture thereof, or a pharmaceutically acceptable salt, solvate, or prodrug thereof:

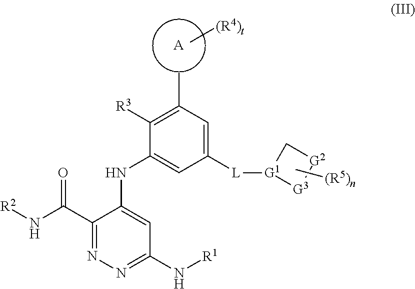

wherein:

ring A, L, G¹, G², G³, R¹ to R⁵, n and t are each as defined in formula (I) above.

In some embodiments of the disclosure, in the compound of formula (I), or a tautomer, mesomer, racemate, enantiomer, diastereomer, or mixture thereof, or a pharmaceutically acceptable salt, solvate, or prodrug thereof, wherein R¹ is —C(O)R⁷, R⁷ is as defined in formula (I) above.

In some embodiments of the disclosure, in the compound of formula (I), or a tautomer, mesomer, racemate, enantiomer, diastereomer, or mixture thereof, or a pharmaceutically acceptable salt, solvate, or prodrug thereof, wherein L is bond or O atom.

In some embodiments of the disclosure, the compound of formula (I), or a tautomer, mesomer, racemate, enantiomer, diastereomer, or mixture thereof, or a pharmaceutically acceptable salt, solvate, or prodrug thereof, is a compound of (IV) or formula (V), or a tautomer, mesomer, racemate, enantiomer, diastereomer, or mixture thereof, or a pharmaceutically acceptable salt, solvate, or prodrug thereof:

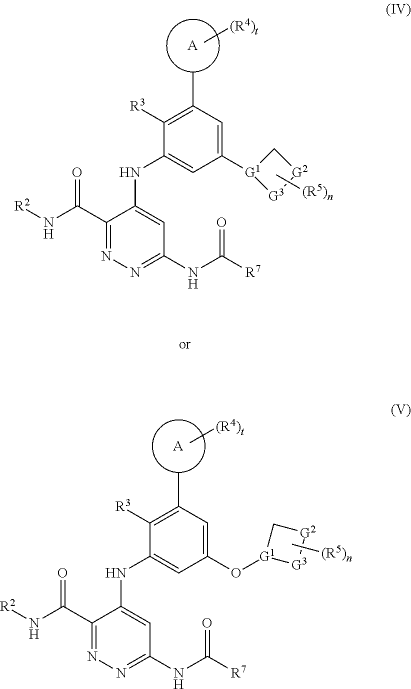

wherein:

ring A, G¹, G², G³, R² to R⁵, R⁷, n and t are each as defined in formula (I).

In some embodiments of the disclosure, the compound of formula (V), or a tautomer, mesomer, racemate, enantiomer, diastereomer, or mixture thereof, or a pharmaceutically acceptable salt, solvate, or prodrug thereof, wherein

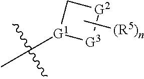

is selected from the group consisting of

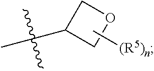

R⁵ is as defined in formula (I).

In some embodiments of the disclosure, the compound of formula (I), or a tautomer, mesomer, racemate, enantiomer, diastereomer, or mixture thereof, or a pharmaceutically acceptable salt, solvate, or prodrug thereof, wherein

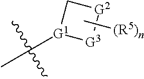

is selected from the group consisting of

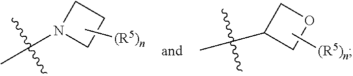

R⁵ is as defined in formula (I).

In some embodiments of the disclosure, the compound of formula (I), or a tautomer, mesomer, racemate, enantiomer, diastereomer, or mixture thereof, or a pharmaceutically acceptable salt, solvate, or prodrug thereof, wherein R⁵ is each identical or different, and each is independently selected from the group consisting of hydrogen, halogen, C₁₋₆alkyl, oxo, C₁₋₆alkoxy, haloC₁₋₆alkyl, haloC₁₋₆alkoxy, hydroxyl and hydroxy C₁₋₆alkyl.

In some embodiments of the disclosure, the compound of formula (I), or a tautomer, mesomer, racemate, enantiomer, diastereomer, or mixture thereof, or a pharmaceutically acceptable salt, solvate, or prodrug thereof, wherein

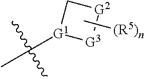

is selected from the group consisting of

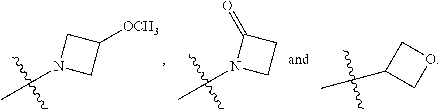 [SMILES: *N1CC(OC)C1 ; *N1CCC1=O ; *C1COC1]

In some embodiments of the disclosure, in the compound of formula (I), or a tautomer, mesomer, racemate, enantiomer, diastereomer, or mixture thereof, or a pharmaceutically acceptable salt, solvate, or prodrug thereof, wherein ring A is 5-member heteroaryl; and preferably triazolyl.

In some embodiments of the disclosure, in the compound of formula (I), or a tautomer, mesomer, racemate, enantiomer, diastereomer, or mixture thereof, or a pharmaceutically acceptable salt, solvate, or prodrug thereof, wherein R² is C₁₋₆alkyl or deuterated C₁₋₆alkyl.

In some embodiments of the disclosure, in the compound of formula (I), or a tautomer, mesomer, racemate, enantiomer, diastereomer, or mixture thereof, or a pharmaceutically acceptable salt, solvate, or prodrug thereof, wherein R³ is C₁₋₆alkoxy.

In some embodiments of the disclosure, in the compound of formula (I), or a tautomer, mesomer, racemate, enantiomer, diastereomer, or mixture thereof, or a pharmaceutically acceptable salt, solvate, or prodrug thereof, wherein R⁴ is each identical or different, and each is independently selected from the group consisting of hydrogen and C₁₋₆alkyl.

In some embodiments of the disclosure, in the compound of formula (I), or a tautomer, mesomer, racemate, enantiomer, diastereomer, or mixture thereof, or a pharmaceutically acceptable salt, solvate, or prodrug thereof, wherein R⁷ is C₃₋₆cycloalkyl; preferably cyclopropyl.

Exemplified compounds of the disclosure include, but are not limited to:

| Ex- | |
|---|---|
| ample | |
| No. | Compound structure and name |
| A1 | 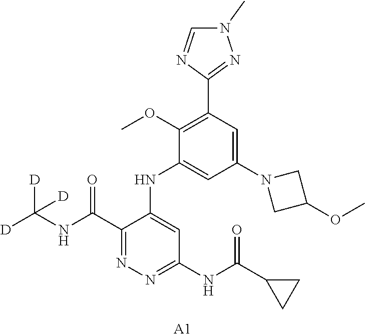 [SMILES: [2H]C([2H])([2H])NC(=O)c1nnc(NC(=O)C2CC2)cc1Nc1cc(N2CC(OC)C2)cc(-c2ncn(C)n2)c1OC ; "A1"] |
| | 6-(cyclopropanecarboxamido)-4-((2-methoxy-5-(3-methoxy- |
| | azetidin-1-yl)-3-(1-methyl-1H-1,2,4-triazol-3-yl)phenyl) |
| | amino)-N-(methyl-d₃)pyridazine-3-carboxamide A1 |
| A2 | 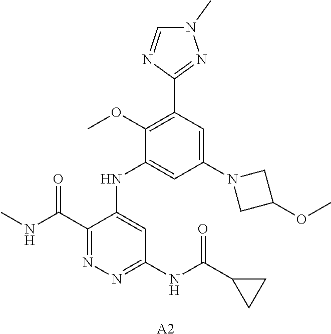 |
| | 6-(cyclopropanecarboxamido)-4-((2-methoxy-5-(3-methoxy- |
| | azetidin-1-yl)-3-(1-methyl-1H-1,2,4-triazol-3-yl)phenyl) |
| | amino)-N-methylpyridazine-3-carboxamide A2 |
| A3 | 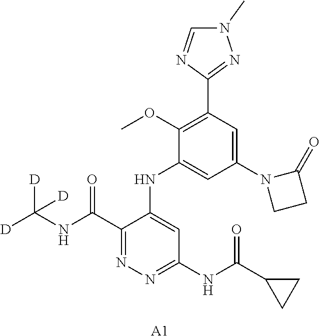 [SMILES: [2H]C([2H])([2H])NC(=O)c1nnc(NC(=O)C2CC2)cc1Nc1cc(N2CCC2=O)cc(-c2ncn(C)n2)c1OC ; "A1"] |
| | 6-(cyclopropanecarboxamido)-4-((2-methoxy-3-(1-methyl-1H- |
| | 1,2,4-triazol-3-yl)-5-(2-oxoazetidin-1-yl)phenyl)amino)-N- |
| | (methyl-d₃)pyridazine-3-carboxamide A3 |
| A4 | 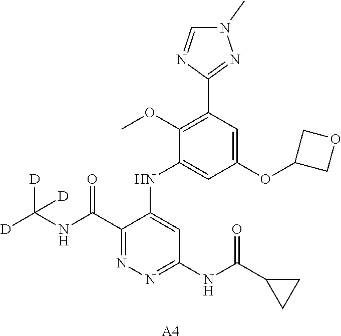 [SMILES: [2H]C([2H])([2H])NC(=O)c1nnc(NC(=O)C2CC2)cc1Nc1cc(OC2COC2)cc(-c2ncn(C)n2)c1OC ; "A4"] |
| | 6-(cyclopropanecarboxamido)-4-((2-methoxy-3-(1-methyl-1H- |
| | 1,2,4-triazol-3-yl)-5-(oxetan-3-yloxy)phenyl)amino)-N- |
| | (methyl-d₃)pyridazine-3-carboxamide A4 |

In another aspect, this disclosure provides a compound of formula (IA), or a tautomer, mesomer, racemate, enantiomer, diastereomer, or mixture thereof, or a pharmaceutically acceptable salt, solvate, or prodrug thereof:

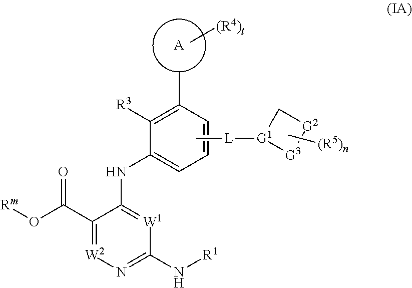

wherein:

R^{m} is hydrogen or alkyl; preferably C₁₋₆ alkyl;

ring A, L, G¹, G², G³, W¹, W², R¹, R³ to R⁵, n and t are each as defined in formula (I).

Exemplified intermediate compounds of the disclosure include, but are not limited to:

| Ex- | |
|---|---|
| ample | |
| No. | Compound structure and name |
| A1a | 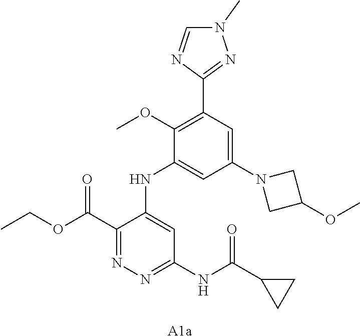 [SMILES: CCOC(=O)c1nnc(NC(=O)C2CC2)cc1Nc1cc(N2CC(OC)C2)cc(-c2ncn(C)n2)c1OC ; "A1a"] |
| | Ethyl 6-(cyclopropanecarboxamido)-4-((2-methoxy-5-(3- |
| | methoxyazetidin-1-yl)-3-(1-methyl-1H-1,2,4-triazol-3- |
| | yl)phenyl)amino)pyridazine-3-carboxylate A1a |
| A3e | 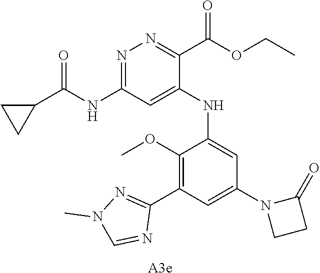 |
| | Ethyl 6-(cyclopropanecarboxamido)-4-((2-methoxy-3-(1- |
| | methyl-1H-1,2,4-triazol-3-yl)-5-(2-oxoazetidin-1-yl)phenyl) |
| | amino)pyridazine-3-carboxylate A3e |
| A4c | 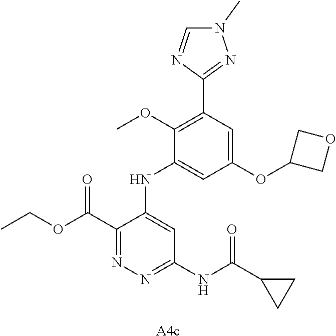 [SMILES: CCOC(=O)c1nnc(NC(=O)C2CC2)cc1Nc1cc(OC2COC2)cc(-c2ncn(C)n2)c1OC ; "A4c"] |
| | Ethyl 6-(cyclopropanecarboxamido)-4-((2-methoxy-3-(1- |
| | methyl-1H-1,2,4-triazol-3-yl)-5-(oxetan-3-yloxy)phenyl) |
| | amino)pyridazine-3-carboxylate A4c |

In another aspect, this disclosure provides a process of preparing the compound of formula (I), or a tautomer, mesomer, racemate, enantiomer, diastereomer, or mixture thereof, or a pharmaceutically acceptable salt, solvate, or prodrug thereof, comprising a step of:

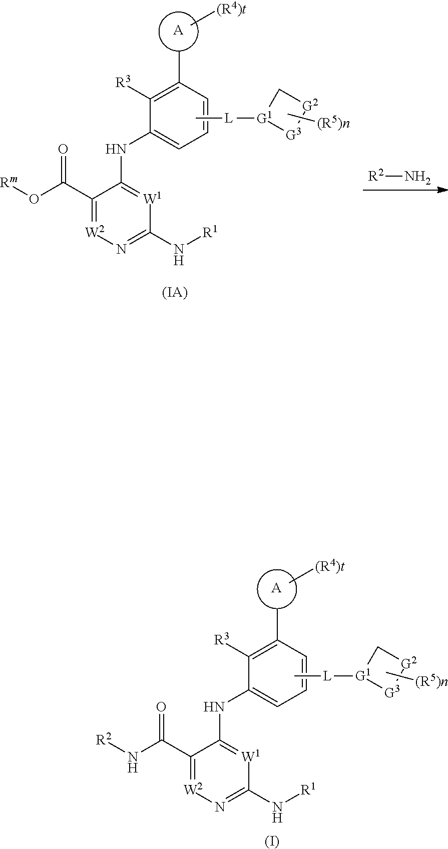

reacting a compound of formula (IA) with a compound of R²—NH₂ to obtain the compound of formula (I);

wherein:

R^{m} is hydrogen or alkyl; preferably C₁₋₆ alkyl;

ring A, L, G¹, G², G³, W¹, W², R¹ to R⁵, n and t are each as defined in formula (I) above.

In another aspect, this disclosure provides a process of preparing the compound of formula (II), or a tautomer, mesomer, racemate, enantiomer, diastereomer, or mixture thereof, or a pharmaceutically acceptable salt, solvate, or prodrug thereof, comprising a step of:

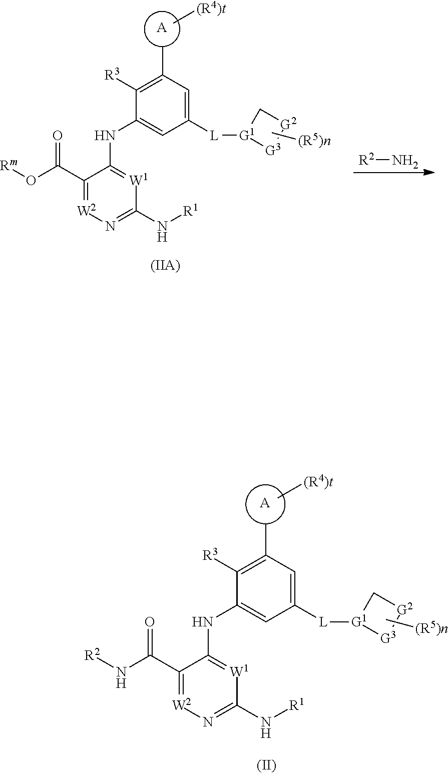

reacting a compound of formula (IIA) with a compound of R²—NH₂ to obtain the compound of formula (II);

wherein:

R^{m} is hydrogen or alkyl; preferably C¹⁻⁶ alkyl;

ring A, L, G¹, G², G³, W¹, W², R¹ to R⁵, n and t are each as defined in formula (II) above.

In another aspect, this disclosure provides a process of preparing the compound of formula (III), or a tautomer, mesomer, racemate, enantiomer, diastereomer, or mixture thereof, or a pharmaceutically acceptable salt, solvate, or prodrug thereof, comprising a step of:

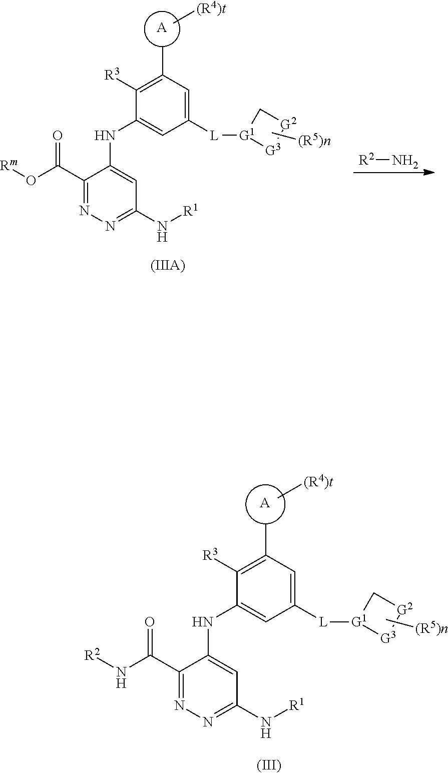

reacting a compound of formula (IIIA) with a compound of R²—NH₂ to obtain the compound of formula (III);

wherein:

R^{m} is hydrogen or alkyl; preferably C₁₋₆ alkyl;

ring A, L, G¹, G², G³, R¹ to R⁵, n and t are each as defined in formula (III) above.

In another aspect, this disclosure provides a process of preparing the compound of formula (IV), or a tautomer, mesomer, racemate, enantiomer, diastereomer, or mixture thereof, or a pharmaceutically acceptable salt, solvate, or prodrug thereof, comprising a step of:

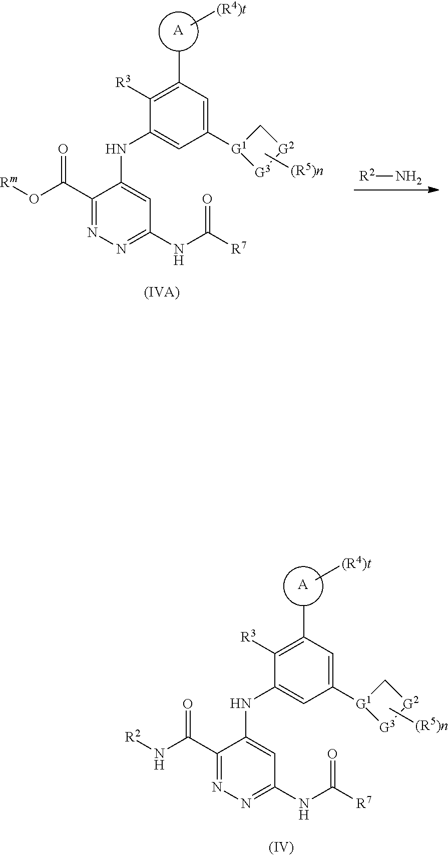

reacting a compound of formula (IVA) with a compound of R²—NH₂ to obtain the compound of formula (IV);

wherein:

R^{m} is hydrogen or alkyl; preferably C₁₋₆ alkyl;

ring A, G¹, G², G³, R² to R⁵, R⁷, n and t are each as defined in formula (I) above.

In another aspect, this disclosure provides a process of preparing the compound of formula (V), or a tautomer, mesomer, racemate, enantiomer, diastereomer, or mixture thereof, or a pharmaceutically acceptable salt, solvate, or prodrug thereof, comprising a step of:

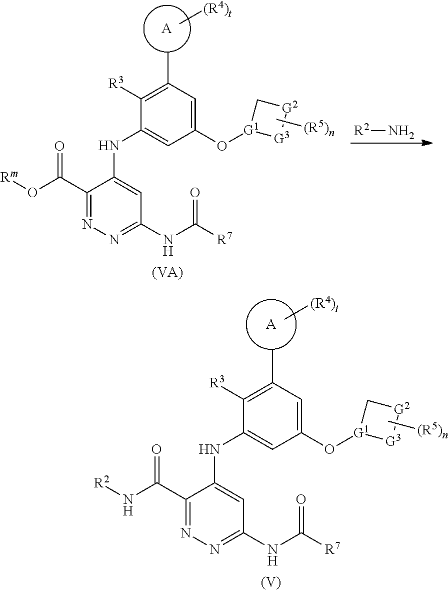

reacting a compound of formula (VA) with a compound of R²—NH₂ to obtain the compound of formula (V);

wherein:

R^{m} is hydrogen or alkyl; preferably C₁₋₆ alkyl;

ring A, G¹, G², G³, R² to R⁵, R⁷, n and t are each as defined in formula (V) above.

The present disclosure also provides a pharmaceutical composition, comprising a therapeutically effective amount of a compound of formula (I), or a tautomer, mesomer, racemate, enantiomer, diastereomer, or mixture thereof, or a pharmaceutically acceptable salt, solvate, or prodrug thereof, together with one or more pharmaceutically acceptable carriers, diluents or excipients.

The present disclosure relates to a method of treating an Tyk2-mediated disorder, disease, or condition, comprising a step of administering to a subject in need thereof a therapeutically effective amount of the compound of formula (I), or a tautomer, mesomer, racemate, enantiomer, diastereomer, or mixture thereof, or a pharmaceutically acceptable salt, solvate, or prodrug thereof, or pharmaceutical composition containing the same.

The present disclosure relates to a method of treating proliferative, metabolic, allergic, autoimmune and inflammatory diseases, comprising a step of administering to a subject in need thereof a therapeutically effective amount of the compound of formula (I), or a tautomer, mesomer, racemate, enantiomer, diastereomer, or mixture thereof, or a pharmaceutically acceptable salt, solvate, or prodrug thereof, or pharmaceutical composition containing the same.

The present disclosure relates to a method of treating autoimmune and inflammatory diseases, comprising a step of administering to a subject in need thereof a therapeutically effective amount of the compound of formula (I), or a tautomer, mesomer, racemate, enantiomer, diastereomer, or mixture thereof, or a pharmaceutically acceptable salt, solvate, or prodrug thereof, or pharmaceutical composition containing the same. Wherein the autoimmune and inflammatory diseases is selected from arthritis, rheumatoid arthritis, multiple sclerosis, systemic lupus erythematosus, lupus nephritis, cutaneous lupus, inflammatory bowel disease, psoriasis, psoriatic arthritis, Crohn's disease, Sjögren's syndrome, systemic scleroderma, ulcerative colitis, Graves' disease, discoid lupus erythematosus, adult onset stills, systemic onset juvenile idiopathic arthritis, gout, gouty arthritis, type I diabetes, insulin dependent diabetes mellitus, sepsis, septic shock, Shigellosis, pancreatitis, glomerulonephritis, autoimmune gastritis, diabetes, autoimmune hemolytic anemia, autoimmune neutropenia, thrombocytopenia, atopic dermatitis, myasthenia gravis, ankylosing spondylitis, pemphigus vulgaris, Goodpasture's disease, antiphospholipid syndrome, idiopathic thrombocytopenia, ANCA-associated vasculitis, pemphigus, Kawasaki disease, Chronic Inflammatory Demyelinating Polyneuropathy (CIDP), dermatomyositis, polymyositis, uveitis, Guillain-Barre syndrome, autoimmune pulmonary inflammation, autoimmune thyroiditis, autoimmune inflammatory eye disease and chronic demyelinating polyneuropathy.

The present disclosure relates to a method of treating cancer, comprising a step of administering to a subject in need thereof a therapeutically effective amount of the compound of formula (I), or a tautomer, mesomer, racemate, enantiomer, diastereomer, or mixture thereof, or a pharmaceutically acceptable salt, solvate, or prodrug thereof, or pharmaceutical composition containing the same. wherein the cancer can be selected from the group consisting of breast cancer, cervical cancer, colon cancer, lung cancer, gastric cancer, rectal cancer, pancreatic cancer, brain cancer, skin cancer, oral cancer, prostate cancer, bone cancer, kidney cancer, ovarian cancer, bladder cancer, liver cancer, tubal tumor, ovarian tumor, peritoneal tumor, melanoma, glioma, neuroblastoma, hepatocellular carcinoma, papillomatosis, head and neck tumor, leukemia, lymphoma, myeloma and non-small cell lung cancer.

In other words, the present disclosure also relates to use of a compound of formula (I), or a tautomer, mesomer, racemate, enantiomer, diastereomer, or mixture thereof, or a pharmaceutically acceptable salt, solvate, or prodrug thereof, or pharmaceutical composition containing the same, in the preparation of a medicament for the treatment of an Tyk2-mediated disorder, disease, or condition.

In other words, the present disclosure also relates to use of a compound of formula (I), or a tautomer, mesomer, racemate, enantiomer, diastereomer, or mixture thereof, or a pharmaceutically acceptable salt, solvate, or prodrug thereof, or pharmaceutical composition containing the same, in the preparation of a medicament for the treatment of proliferative, metabolic, allergic, autoimmune and inflammatory diseases.

In other words, the present disclosure also relates to use of a compound of formula (I), or a tautomer, mesomer, racemate, enantiomer, diastereomer, or mixture thereof, or a pharmaceutically acceptable salt, solvate, or prodrug thereof, or pharmaceutical composition containing the same, in the preparation of a medicament for the treatment of autoimmune and inflammatory diseases. Wherein the autoimmune and inflammatory diseases is selected from arthritis, rheumatoid arthritis, multiple sclerosis, systemic lupus erythematosus, lupus nephritis, cutaneous lupus, inflammatory bowel disease, psoriasis, psoriatic arthritis, Crohn's disease, Sjögren's syndrome, systemic scleroderma, ulcerative colitis, Graves' disease, discoid lupus erythematosus, adult onset stills, systemic onset juvenile idiopathic arthritis, gout, gouty arthritis, type I diabetes, insulin dependent diabetes mellitus, sepsis, septic shock, Shigellosis, pancreatitis, glomerulonephritis, autoimmune gastritis, diabetes, autoimmune hemolytic anemia, autoimmune neutropenia, thrombocytopenia, atopic dermatitis, myasthenia gravis, ankylosing spondylitis, pemphigus vulgaris, Goodpasture's disease, antiphospholipid syndrome, idiopathic thrombocytopenia, ANCA-associated vasculitis, pemphigus, Kawasaki disease, Chronic Inflammatory Demyelinating Polyneuropathy (CIDP), dermatomyositis, polymyositis, uveitis, Guillain-Barre syndrome, autoimmune pulmonary inflammation, autoimmune thyroiditis, autoimmune inflammatory eye disease and chronic demyelinating polyneuropathy.

In other words, the present disclosure also relates to use of a compound of formula (I), or a tautomer, mesomer, racemate, enantiomer, diastereomer, or mixture thereof, or a pharmaceutically acceptable salt, solvate, or prodrug thereof, or pharmaceutical composition containing the same, in the preparation of a medicament for the treatment of cancer. wherein the cancer can be selected from the group consisting of breast cancer, cervical cancer, colon cancer, lung cancer, gastric cancer, rectal cancer, pancreatic cancer, brain cancer, skin cancer, oral cancer, prostate cancer, bone cancer, kidney cancer, ovarian cancer, bladder cancer, liver cancer, tubal tumor, ovarian tumor, peritoneal tumor, melanoma, glioma, neuroblastoma, hepatocellular carcinoma, papillomatosis, head and neck tumor, leukemia, lymphoma, myeloma and non-small cell lung cancer.

The present disclosure further relates to the compound of formula (I), or a tautomer, mesomer, racemate, enantiomer, diastereomer, or mixture thereof, or a pharmaceutically acceptable salt, solvate, or prodrug thereof, or pharmaceutical composition containing the same, for use as a medicament.

The present disclosure also relates to the compound of formula (I), or a tautomer, mesomer, racemate, enantiomer, diastereomer, or mixture thereof, or a pharmaceutically acceptable salt, solvate, or prodrug thereof, or pharmaceutical composition containing the same, for use in treating an Tyk2-mediated disorder, disease, or condition.

The present disclosure also relates to the combination of the compound of formula (I), or a tautomer, mesomer, racemate, enantiomer, diastereomer, or mixture thereof, or a pharmaceutically acceptable salt, solvate, or prodrug thereof, or pharmaceutical composition containing the same, for use in treating proliferative, metabolic, allergic, autoimmune and inflammatory diseases.

The present disclosure also relates to the combination of the compound of formula (I), or a tautomer, mesomer, racemate, enantiomer, diastereomer, or mixture thereof, or a pharmaceutically acceptable salt, solvate, or prodrug thereof, or pharmaceutical composition containing the same, for use in treating autoimmune and inflammatory diseases. wherein the autoimmune and inflammatory diseases is selected from arthritis, rheumatoid arthritis, multiple sclerosis, systemic lupus erythematosus, lupus nephritis, cutaneous lupus, inflammatory bowel disease, psoriasis, psoriatic arthritis, Crohn's disease, Sjögren's syndrome, systemic scleroderma, ulcerative colitis, Graves' disease, discoid lupus erythematosus, adult onset stills, systemic onset juvenile idiopathic arthritis, gout, gouty arthritis, type I diabetes, insulin dependent diabetes mellitus, sepsis, septic shock, Shigellosis, pancreatitis, glomerulonephritis, autoimmune gastritis, diabetes, autoimmune hemolytic anemia, autoimmune neutropenia, thrombocytopenia, atopic dermatitis, myasthenia gravis, ankylosing spondylitis, pemphigus vulgaris, Goodpasture's disease, antiphospholipid syndrome, idiopathic thrombocytopenia, ANCA-associated vasculitis, pemphigus, Kawasaki disease, Chronic Inflammatory Demyelinating Polyneuropathy (CIDP), dermatomyositis, polymyositis, uveitis, Guillain-Barre syndrome, autoimmune pulmonary inflammation, autoimmune thyroiditis, autoimmune inflammatory eye disease and chronic demyelinating polyneuropathy.

The present disclosure also relates to the combination of the compound of formula (I), or a tautomer, mesomer, racemate, enantiomer, diastereomer, or mixture thereof, or a pharmaceutically acceptable salt, solvate, or prodrug thereof, or pharmaceutical composition containing the same, for use in treating cancer. wherein the cancer can be selected from the group consisting of breast cancer, cervical cancer, colon cancer, lung cancer, gastric cancer, rectal cancer, pancreatic cancer, brain cancer, skin cancer, oral cancer, prostate cancer, bone cancer, kidney cancer, ovarian cancer, bladder cancer, liver cancer, tubal tumor, ovarian tumor, peritoneal tumor, melanoma, glioma, neuroblastoma, hepatocellular carcinoma, papillomatosis, head and neck tumor, leukemia, lymphoma, myeloma and non-small cell lung cancer.

The active compounds can be formulated in a form suitable for administration by any suitable route using conventional methods using one or more pharmaceutically acceptable carriers. Therefore, the active compound of the present disclosure can be formulated into various dosage forms for oral administration, injection (for example, intravenous, intramuscular or subcutaneous) administration, inhalation or insufflation. The compounds of the disclosure may also be formulated in sustained release dosage forms, such as tablets, hard or soft capsules, aqueous or oily suspensions, emulsions, injections, dispersible powders or granules, suppositories, troches, or syrups.

The dosage of the compound or composition used in the method of treatment of the disclosure will generally vary with the severity of the disease, the weight of the patient and the relative efficacy of the compound. However, as a general guide, the active compound is preferably in the form of a unit dose or in such a way that the patient can self-administer in a single dose. The unit dose of the compound or composition of the present disclosure can be expressed in the form of tablets, capsules, cachets, bottled portions, powders, granules, lozenges, suppositories, reconstituted powders or liquid preparations. A suitable unit dose may be from 0.1 to 1000 mg.

In addition to the active compound, the pharmaceutical composition of the present disclosure may contain one or more excipients selected from the following ingredients: fillers (diluents), binders, wetting agents, disintegrating agents, or excipients. Depending on the method of administration, the composition may contain from 0.1 to 99%, sometimes preferably 1 to 70%, by weight of active compound.

Tablets contain the active ingredients and non-toxic pharmaceutically acceptable excipients suitable for the preparation of tablets for mixing. These excipients can be inert excipients, granulating agents, disintegrating agents, binders and lubricants. These tablets can be uncoated or they can be coated by known techniques that mask the taste of the drug or delay disintegration and absorption in the gastrointestinal tract, thereby providing a sustained release over a longer period.

Oral formulations may also be provided in soft gelatin capsules in which the active ingredient is mixed with an inert solid diluent or in which the active ingredient is mixed with a water-soluble carrier or an oil vehicle.

Aqueous suspensions contain the active substance and excipients suitable for the preparation of the aqueous suspension for mixing. Such excipients are suspending, dispersing or wetting agents. The aqueous suspension may also contain one or more preservatives, one or more colorants, one or more flavoring agents, and one or more sweetening agents.

Oil suspensions can be formulated by suspending the active ingredient in a vegetable or mineral oil. The oil suspension may contain a thickener. The sweeteners and flavoring agents described above can be added to provide a palatable formulation. These compositions can be preserved by the addition of antioxidants.

The pharmaceutical composition of the present disclosure may also be in the form of an oil-in-water emulsion. The oil phase may be a vegetable oil, or a mineral oil or a mixture thereof. Suitable emulsifiers may be naturally occurring phospholipids, and emulsions may also contain sweeteners, flavoring agents, preservatives and antioxidants. Such formulations may also contain a demulcent, a preservative, a colorant, and an antioxidant.

The pharmaceutical composition of the present disclosure may be in the form of a sterile injectable aqueous solution. Among the acceptable vehicles or solvents that may be employed are water, Ringer's solution and isotonic sodium chloride solution. The sterile injectable preparation may be a sterile injectable oil-in-water microemulsion in which the active ingredient is dissolved in the oil phase, and the injection solution or microemulsion may be injected into the bloodstream of the patient by local large-scale injection. Alternatively, solutions and microemulsions are preferably administered in a manner that maintains a constant circulating concentration of the compounds of the present disclosure. To maintain this constant concentration, continuous intravenous drug delivery devices can be used. An example of such a device is the Deltec CADD-PLUS™ 5400 intravenous pump.

The pharmaceutical composition of the present disclosure may be in the form of a sterile injectable water or oily suspension for intramuscular and subcutaneous administration. This suspension may be formulated according to the known art using those suitable dispersing or wetting agents and suspending agents which have been mentioned above. The sterile injectable preparation may also be a sterile injectable solution or suspension prepared in a parenterally acceptable non-toxic diluent or solvent. In addition, a sterile fixed oil can be conveniently used as a solvent or suspension medium. For this purpose, any blending fixing oil can be used. In addition, fatty acids can also be prepared for injection.

The compounds of the present disclosure can be administered in the form of suppositories for rectal administration. These pharmaceutical compositions can be prepared by mixing the drug with a suitable non-irritating excipient that is solid at ordinary temperatures but liquid in the rectum and therefore will dissolve in the rectum to release the drug.

Compounds of the present disclosure can be administered by adding water to prepare water-suspendable dispersible powders and granules. These pharmaceutical compositions can be prepared by mixing the active ingredient with a dispersing or wetting agent, a suspending agent or one or more preservatives.

As is well known to those skilled in the art, the dosage of a drug depends on a number of factors, including but not limited to the following: the activity of the specific compound used, the age of the patient, the weight of the patient, the patient's health, and the patient's behavior, The patient's diet, the time of administration, the manner of administration, the rate of excretion, the combination of drugs, etc.; in addition, the best treatment such as the mode of treatment, the daily dosage of the general compound (I) or the pharmaceutically acceptable salt, solvate, or prodrug thereof. The type can be verified according to the traditional treatment plan.

### Definitions

Unless otherwise stated, the terms used in the specification and claims have the meanings described below.

“Alkyl” refers to a saturated aliphatic hydrocarbon group including C₁-C₂₀ straight chain and branched chain groups. Preferably an alkyl group is an alkyl having 1 to 12 carbon atoms. In some embodiments, sometimes preferably, an alkyl group is an alkyl having 1 to 8 carbon atoms. Representative examples include, but are not limited to methyl, ethyl, n-propyl, isopropyl, n-butyl, isobutyl, tert-butyl, sec-butyl, n-pentyl, 1,1-dimethyl propyl, 1,2-dimethyl propyl, 2,2-dimethyl propyl, 1-ethyl propyl, 2-methylbutyl, 3-methylbutyl, n-hexyl, 1-ethyl-2-methylpropyl, 1,1,2-trimethylpropyl, 1,1-dimethylbutyl, 1,2-dimethylbutyl, 2,2-dimethylbutyl, 1,3-dimethylbutyl, 2-ethylbutyl, 2-methylpentyl, 3-methylpentyl, 4-methylpentyl, 2,3-dimethylbutyl, n-heptyl, 2-methylhexyl, 3-methylhexyl, 4-methylhexyl, 5-methylhexyl, 2,3-dimethylpentyl, 2,4-dimethylpentyl, 2,2-dimethylpentyl, 3,3-dimethylpentyl, 2-ethylpentyl, 3-ethylpentyl, n-octyl, 2,3-dimethylhexyl, 2,4-dimethylhexyl, 2,5-dimethylhexyl, 2,2-dimethylhexyl, 3,3-dimethylhexyl, 4,4-dimethylhexyl, 2-ethylhexyl, 3-ethylhexyl, 4-ethylhexyl, 2-methyl-2-ethylpentyl, 2-methyl-3-ethylpentyl, n-nonyl, 2-methyl-2-ethylhexyl, 2-methyl-3-ethylhexyl, 2,2-diethylpentyl, n-decyl, 3,3-diethylhexyl, 2,2-diethylhexyl, and the isomers of branched chain thereof. In some embodiments, sometimes more preferably an alkyl group is a lower alkyl having 1 to 6 carbon atoms, and sometimes more preferably 1 to 4 carbon atoms. Representative examples include, but are not limited to, methyl, ethyl, n-propyl, isopropyl, n-butyl, isobutyl, tert-butyl, sec-butyl, n-pentyl, 1,1-dimethylpropyl, 1,2-dimethylpropyl, 2,2-dimethylpropyl, 1-ethylpropyl, 2-methylbutyl, 3-methylbutyl, n-hexyl, 1-ethyl-2-methylpropyl, 1,1,2-trimethylpropyl, 1,1-dimethylbutyl, 1,2-dimethylbutyl, 2,2-dimethylbutyl, 1,3-dimethylbutyl, 2-ethylbutyl, 2-methylpentyl, 3-methylpentyl, 4-methylpentyl, 2,3-dimethylbutyl, etc. The alkyl group can be substituted or unsubstituted. When substituted, the substituent group(s) can be substituted at any available connection point, preferably the substituent group(s) is one or more, sometimes preferably 1 to 5, and sometimes more preferably 1 to 3, groups independently selected from the group consisting of alkyl, halogen, alkoxy, alkenyl, alkynyl, alkylsulfo, alkylamino, thiol, hydroxyl, nitro, cyano, amino, cycloalkyl, heterocyclic alkyl, aryl, heteroaryl, cycloalkoxyl, heterocylic, cycloalkylthio, heterocylic alkylthio and oxo group.

“Alkenyl” refers to an alkyl defined as above that has at least two carbon atoms and at least one carbon-carbon double bond, for example, vinyl, 1-propenyl, 2-propenyl, 1-, 2-, or 3-butenyl, etc., preferably C₂₋₂₀ alkenyl, more preferably C₂₋₁₂ alkenyl, and sometimes more preferably C₂₋₆ alkenyl, and sometimes even more preferably C₂₋₄ alkenyl. The alkenyl group can be substituted or unsubstituted. When substituted, the substituent group(s) is preferably one or more, sometimes preferably 1 to 5, and sometimes more preferably 1 to 3, group(s) independently selected from the group consisting of alkyl, halogen, alkoxy, alkenyl, alkynyl, alkylsulfo, alkylamino, thiol, hydroxyl, nitro, cyano, amino, cycloalkyl, heterocyclic alkyl, aryl, heteroaryl, cycloalkoxyl, heterocylic, cycloalkylthio, heterocylic alkylthio and oxo group.

“Alkynyl” refers to an alkyl defined as above that has at least two carbon atoms and at least one carbon-carbon triple bond, for example, ethynyl, 1-propynyl, 2-propynyl, 1-, 2-, or 3-butynyl etc., preferably C₂₋₂₀ alkynyl, more preferably C₂₋₁₂ alkynyl, and sometimes preferably C₂₋₆ alkynyl, and sometimes even more preferably C₂₋₄ alkynyl. The alkynyl group can be substituted or unsubstituted. When substituted, the substituent group(s) is preferably one or more, sometimes preferably 1 to 5, and sometimes more preferably 1 to 3, group(s) independently selected from the group consisting of alkyl, alkenyl, alkynyl, alkoxy, alkylsulfo, alkylamino, halogen, thiol, hydroxyl, nitro, cyano, cycloalkyl, heterocyclic alkyl, aryl, heteroaryl, cycloalkoxyl, heterocylic alkoxyl, cycloalkylthio and heterocylic alkylthio.

“Alkylene” refers to a saturated linear or branched divalent aliphatic hydrocarbon group, derived by removing two hydrogen atoms from the same carbon atom or two different carbon atoms of the parent alkane. The straight or branched chain group contains 1 to 20 carbon atoms, preferably 1 to 12 carbon atoms, sometimes more preferably 1 to 6 carbon atoms, and sometimes more preferably 1 to 4 carbon atoms. Non-limiting examples of alkylene groups include, but are not limited to, methylene (—CH₂—), 1,1-ethylene (—CH(CH₃)—), 1,2-ethylene (—CH₂CH₂)—, 1,1-propylene (—CH(CH₂CH₃)—), 1,2-propylene (—CH₂CH(CH₃)—), 1,3-propylene (—CH₂CH₂CH₂—), 1,4-butylidene (—CH₂CH₂CH₂CH₂—), etc. The alkylene group can be substituted or unsubstituted. When substituted, the substituent group(s) is preferably one or more, sometimes preferably 1 to 5, and sometimes more preferably 1 to 3, group(s) independently selected from the group consisting of selected from alkyl, alkenyl, alkynyl, alkoxy, alkylsulfo, alkylamino, halogen, thiol, hydroxyl, nitro, cyano, cycloalkyl, heterocyclic alkyl, aryl, heteroaryl, cycloalkoxyl, heterocylic alkoxyl, cycloalkylthio and heterocylic alkylthio.

“Alkenylene” refers to an alkylene defined as above that has at least two carbon atoms and at least one carbon-carbon double bond, preferably C₂₋₂₀ alkenylene, more preferably C₂₋₁₂ alkenylene, sometimes more preferably C₂₋₆ alkenylene, and sometimes even more preferably C₂₋₄ alkenylene. Non-limiting examples of alkenylene groups include, but are not limited to, —CH═CH—, —CH═CHCH₂—, —CH═CHCH₂CH₂—, —CH₂CH═CHCH₂—, etc. The alkenylene group can be substituted or unsubstituted. When substituted, the substituent group(s) is preferably one or more, sometimes preferably 1 to 5, and sometimes more preferably 1 to 3, group(s) independently selected from the group consisting of alkyl, alkenyl, alkynyl, alkoxy, alkylsulfo, alkylamino, halogen, thiol, hydroxyl, nitro, cyano, cycloalkyl, heterocyclic alkyl, aryl, heteroaryl, cycloalkoxyl, heterocylic alkoxyl, cycloalkylthio and heterocylic alkylthio.

“Cycloalkyl” refers to a saturated and/or partially unsaturated monocyclic or polycyclic hydrocarbon group having 3 to 20 carbon atoms, preferably 3 to 12 carbon atoms, more preferably 3 to 10 carbon atoms, sometimes more preferably 3 to 8 (for example 3, 4, 5, 6, 7 or 8) carbon atoms, and sometimes even more preferably 3 to 6 carbon atoms. Representative examples of monocyclic cycloalkyls include, but are not limited to, cyclopropyl, cyclobutyl, cyclopentyl, cyclopentenyl, cyclohexyl, cyclohexenyl, cyclohexadienyl, cycloheptyl, cycloheptatrienyl, cyclooctyl, etc. Polycyclic cycloalkyl includes a cycloalkyl having a spiro ring, fused ring or bridged ring.

“Spiro Cycloalkyl” refers to a 5 to 20 membered polycyclic group with rings connected through one common carbon atom (called a spiro atom), wherein one or more rings can contain one or more double bonds, but none of the rings has a completely conjugated pi-electron system. Preferably a spiro cycloalkyl is 6 to 14 membered, and more preferably 7 to 10 (for example 7, 8, 9 or 10) membered. According to the number of common spiro atoms, a spiro cycloalkyl is divided into mono-spiro cycloalkyl, di-spiro cycloalkyl, or poly-spiro cycloalkyl, and preferably refers to a mono-spiro cycloalkyl or di-spiro cycloalkyl, more preferably 4-membered/4-membered, 4-membered/5-membered, 4-membered/6-membered, 5-membered/5-membered, or 5-membered/6-membered mono-spiro cycloalkyl. Representative examples of spiro cycloalkyl include, but are not limited to the following groups:

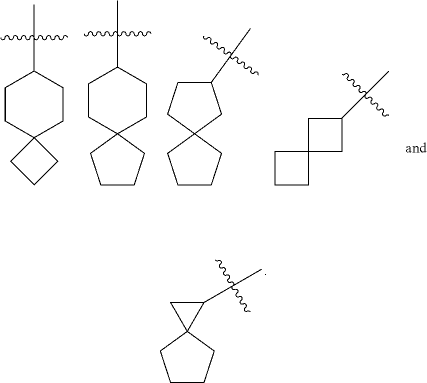

“Fused Cycloalkyl” refers to a 5 to 20 membered polycyclic hydrocarbon group, wherein each ring in the system shares an adjacent pair of carbon atoms with another ring, wherein one or more rings can contain one or more double bonds, but none of the rings has a completely conjugated pi-electron system. Preferably, a fused cycloalkyl group is 6 to 14 membered, more preferably 7 to 10 (for example 7, 8, 9 or 10) membered. According to the number of membered rings, fused cycloalkyl is divided into bicyclic, tricyclic, tetracyclic or polycyclic fused cycloalkyl, and preferably refers to a bicyclic or tricyclic fused cycloalkyl, more preferably 5-membered/5-membered, or 5-membered/6-membered bicyclic fused cycloalkyl. Representative examples of fused cycloalkyls include, but are not limited to, the following groups:

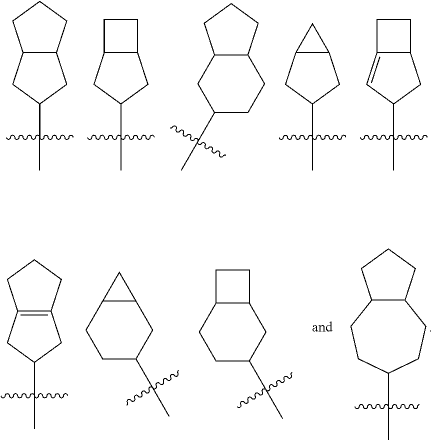

“Bridged Cycloalkyl” refers to a 5 to 20 membered polycyclic hydrocarbon group, wherein every two rings in the system share two disconnected carbon atoms. The rings can have one or more double bonds, but have no completely conjugated pi-electron system. Preferably, a bridged cycloalkyl is 6 to 14 membered, and more preferably 7 to 10 (for example 7, 8, 9 or 10) membered. According to the number of membered rings, bridged cycloalkyl is divided into bicyclic, tricyclic, tetracyclic or polycyclic bridged cycloalkyl, and preferably refers to a bicyclic, tricyclic or tetracyclic bridged cycloalkyl, more preferably a bicyclic or tricyclic bridged cycloalkyl. Representative examples of bridged cycloalkyls include, but are not limited to, the following groups:

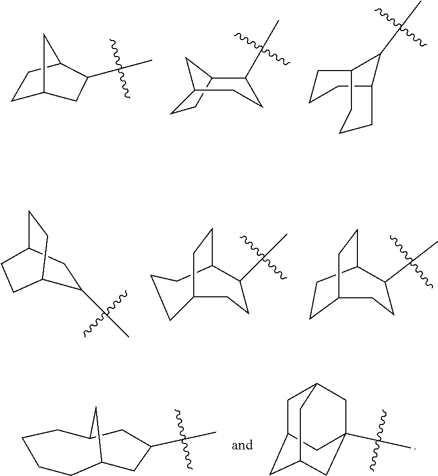

The cycloalkyl also includes the cycloalkyl said above which fused to the ring of an aryl, heteroaryl or heterocyclic alkyl, wherein the ring bound to the parent structure is cycloalkyl. Representative examples include, but are not limited to indanylacetic, tetrahydronaphthalene, benzocycloheptyl and so on. The cycloalkyl is optionally substituted or unsubstituted. When substituted, the substituent group(s) is preferably one or more, sometimes preferably 1 to 5, and sometimes more preferably 1 to 3, groups independently selected from the group consisting of alkyl, halogen, alkoxy, alkenyl, alkynyl, alkylsulfo, alkylamino, thiol, hydroxyl, nitro, cyano, amino, cycloalkyl, heterocyclic alkyl, aryl, heteroaryl, cycloalkoxyl, heterocylic, cycloalkylthio, heterocylic alkylthio and oxo group.

“Heterocyclyl” refers to a 3 to 20 membered saturated and/or partially unsaturated monocyclic or polycyclic hydrocarbon group having one or more heteroatoms selected from the group consisting of N, O, S, S(O) and S(O)₂ as ring atoms, but excluding —O—O—, —O—S— or —S—S— in the ring, the remaining ring atoms being C. Preferably, heterocyclyl is a 3 to 12 membered having 1 to 4 heteroatoms; more preferably a 3 to 10 (for example 3, 4, 5, 6, 7, 8, 9 or 10) membered having 1 to 3 heteroatoms; more preferably a 6 to 10 membered having 1 to 3 heteroatoms; most preferably a 5 to 6 membered having 1 to 2 heteroatoms. Representative examples of monocyclic heterocyclyls include, but are not limited to, pyrrolidyl, piperidyl, piperazinyl, morpholinyl, sulfo-morpholinyl, homopiperazinyl, and so on. Polycyclic heterocyclyl includes the heterocyclyl having a spiro ring, fused ring or bridged ring.

“Spiro heterocyclyl” refers to a 5 to 20 membered polycyclic heterocyclyl with rings connected through one common carbon atom (called a spiro atom), wherein said rings have one or more heteroatoms selected from the group consisting of N, O, S, S(O) and S(O)₂ as ring atoms, the remaining ring atoms being C, wherein one or more rings can contain one or more double bonds, but none of the rings has a completely conjugated pi-electron system. Preferably a spiro heterocyclyl is 6 to 14 membered, and more preferably 7 to 10 membered. According to the number of common Spiro atoms, Spiro heterocyclyl is divided into mono-Spiro heterocyclyl, di-spiro heterocyclyl, or poly-spiro heterocyclyl, and preferably refers to mono-spiro heterocyclyl or di-spiro heterocyclyl, more preferably 4-membered/4-membered, 4-membered/5-membered, 4-membered/6-membered, 5-membered/5-membered, or 5-membered/6-membered mono-spiro heterocyclyl. Representative examples of spiro heterocyclyl include, but are not limited to the following groups:

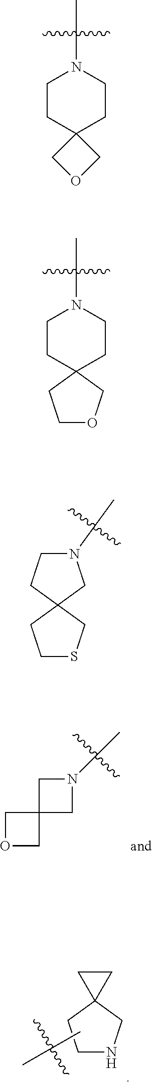

“Fused Heterocyclyl” refers to a 5 to 20 membered polycyclic heterocyclyl group, wherein each ring in the system shares an adjacent pair of carbon atoms with the other ring, wherein one or more rings can contain one or more double bonds, but none of the rings has a completely conjugated pi-electron system, and wherein said rings have one or more heteroatoms selected from the group consisting of N, O, S, S(O) and S(O)₂ as ring atoms, the remaining ring atoms being C. Preferably a fused heterocyclyl is 6 to 14 membered, and more preferably 7 to 10 membered. According to the number of membered rings, fused heterocyclyl is divided into bicyclic, tricyclic, tetracyclic or polycyclic fused heterocyclyl, preferably refers to bicyclic or tricyclic fused heterocyclyl, more preferably 5-membered/5-membered, or 5-membered/6-membered bicyclic fused heterocyclyl. Representative examples of fused heterocyclyl include, but are not limited to, the following groups:

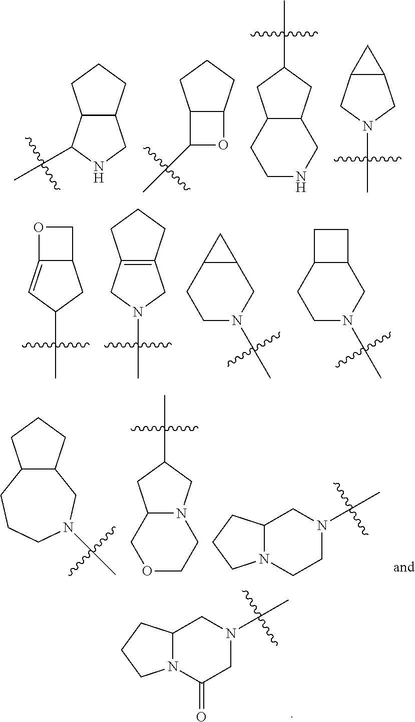

“Bridged Heterocyclyl” refers to a 5 to 14 membered polycyclic heterocyclic alkyl group, wherein every two rings in the system share two disconnected atoms, the rings can have one or more double bonds, but have no completely conjugated pi-electron system, and the rings have one or more heteroatoms selected from the group consisting of N, O S, S(O) and S(O)₂ as ring atoms, the remaining ring atoms being C. Preferably a bridged heterocyclyl is 6 to 14 membered, and more preferably 7 to 10 membered. According to the number of membered rings, bridged heterocyclyl is divided into bicyclic, tricyclic, tetracyclic or polycyclic bridged heterocyclyl, and preferably refers to bicyclic, tricyclic or tetracyclic bridged heterocyclyl, more preferably bicyclic or tricyclic bridged heterocyclyl. Representative examples of bridged heterocyclyl include, but are not limited to, the following groups:

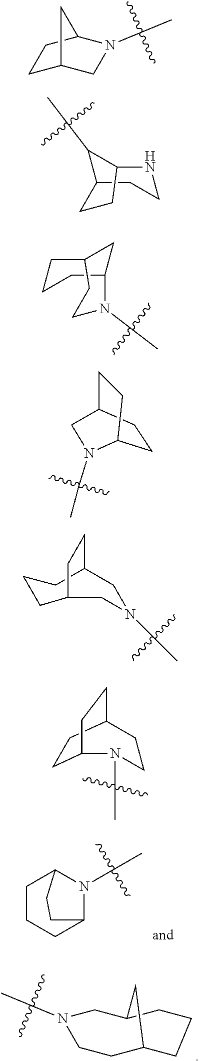

The ring of said heterocyclyl include the heterocyclyl said above which fused to the ring of an aryl, heteroaryl or cycloalkyl, wherein the ring bound to the parent structure is heterocyclyl. Representative examples include, but are not limited to the following groups:

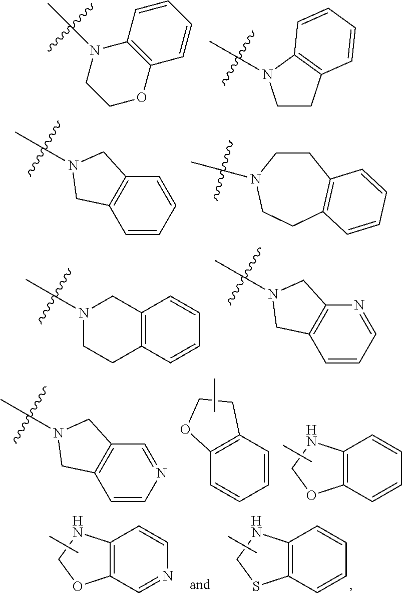

or the like.

The heterocyclyl is optionally substituted or unsubstituted. When substituted, the substituent group(s) is preferably one or more, sometimes preferably 1 to 5, and sometimes more preferably 1 to 3, group(s) independently selected from the group consisting of alkyl, alkenyl, alkynyl, alkoxy, alkylsulfo, alkylamino, halogen, thiol, hydroxyl, nitro, cyano, cycloalkyl, heterocyclic alkyl, aryl, heteroaryl, cycloalkoxyl, heterocylic alkoxyl, cycloalkylthio and heterocylic alkylthio.

“Aryl” refers to a 6 to 14 membered all-carbon monocyclic ring or a polycyclic fused ring (a “fused” ring system means that each ring in the system shares an adjacent pair of carbon atoms with another ring in the system) group, and has a completely conjugated pi-electron system. Preferably aryl is 6 to 10 membered, such as phenyl and naphthyl, most preferably phenyl. The aryl include the aryl said above which fused to the ring of heteroaryl, heterocyclyl or cycloalkyl, wherein the ring bound to parent structure is aryl. Representative examples include, but are not limited to, the following groups:

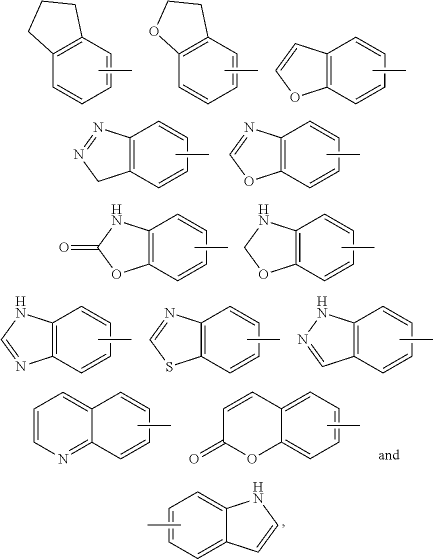

or the like.

The aryl group can be substituted or unsubstituted. When substituted, the substituent group(s) is preferably one or more, sometimes preferably 1 to 5, and sometimes more preferably 1 to 3, groups independently selected from the group consisting of alkyl, alkenyl, alkynyl, alkoxy, alkylsulfo, alkylamino, halogen, thiol, hydroxyl, nitro, cyano, cycloalkyl, heterocyclic alkyl, aryl, heteroaryl, cycloalkoxyl, heterocylic alkoxyl, cycloalkylthio and heterocylic alkylthio.

“Heteroaryl” refers to an aryl system having 1 to 4 heteroatoms selected from the group consisting of O, S and N as ring atoms and having 5 to 14 annular atoms. Preferably a heteroaryl is 5- to 10-membered, more preferably 5- or 6-membered, for example, thiadiazolyl, pyrazolyl, oxazolyl, oxadiazolyl, imidazolyl, triazolyl, thiazolyl, furyl, thienyl, pyridyl, pyrrolyl, N-alkyl pyrrolyl, pyrimidinyl, pyrazinyl, imidazolyl, tetrazolyl, and the like. The heteroaryl include the heteroaryl said above which fused with the ring of an aryl, heterocyclyl or cycloalkyl, wherein the ring bound to parent structure is heteroaryl. Representative examples include, but are not limited to, the following groups:

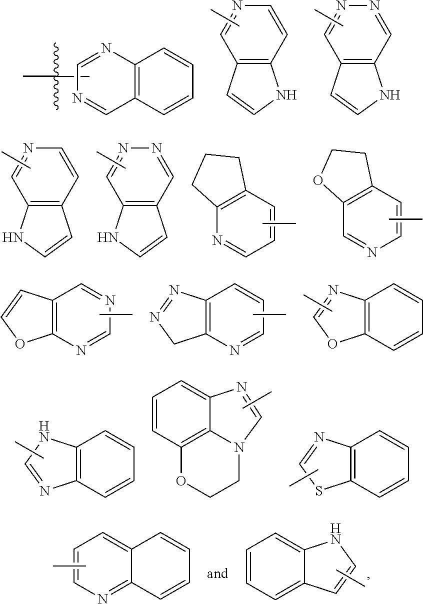

or the like.

The heteroaryl group can be substituted or unsubstituted. When substituted, the substituent group(s) is preferably one or more, sometimes preferably 1 to 5, and sometimes more preferably 1 to 3, groups independently selected from the group consisting of alkyl, alkenyl, alkynyl, alkoxy, alkylsulfo, alkylamino, halogen, thiol, hydroxyl, nitro, cyano, cycloalkyl, heterocyclic alkyl, aryl, heteroaryl, cycloalkoxyl, heterocylic alkoxyl, cycloalkylthio and heterocylic alkylthio.

“Alkoxy” refers to both an —O-(alkyl) and an —O-(unsubstituted cycloalkyl) group, wherein the alkyl is defined as above. Representative examples include, but are not limited to, methoxy, ethoxy, propoxy, butoxy, cyclopropyloxy, cyclobutyloxy, cyclopentyloxy, cyclohexyloxy, and the like. The alkoxyl can be substituted or unsubstituted. When substituted, the substituent is preferably one or more groups, sometimes preferably 1 to 5, and sometimes more preferably 1 to 3, independently selected from the group consisting of alkyl, alkenyl, alkynyl, alkoxy, alkylsulfo, alkylamino, halogen, thiol, hydroxyl, nitro, cyano, cycloalkyl, heterocyclic alkyl, aryl, heteroaryl, cycloalkoxyl, heterocylic alkoxyl, cycloalkylthio and heterocylic alkylthio.

“Bond” refers to a covalent bond using a sign of “-”.

“Hydroxyalkyl” refers to an alkyl group substituted by a hydroxyl group, wherein alkyl is as defined above.

“Deuterated alkyl” refers to an alkyl group substituted by a or more deuterium atom, wherein alkyl is as defined above.

“Hydroxyl” refers to an —OH group.

“Halogen” refers to fluoro, chloro, bromo or iodo atoms.

“Amino” refers to a —NH₂ group.

“Cyano” refers to a —CN group.

“Nitro” refers to a —NO₂ group.

“Oxo group” refers to a ═O group.

“Carboxyl” refers to a —C(O)OH group.

“Alkoxycarbonyl” refers to a —C(O)O(alkyl) or (cycloalkyl) group, wherein the alkyl and cycloalkyl are defined as above.

“Optional” or “optionally” means that the event or circumstance described subsequently can, but need not, occur, and the description includes the instances in which the event or circumstance may or may not occur. For example, “the heterocyclic group optionally substituted by an alkyl” means that an alkyl group can be, but need not be, present, and the description includes the case of the heterocyclic group being substituted with an alkyl and the heterocyclic group being not substituted with an alkyl.

“Substituted” refers to one or more hydrogen atoms in the group, preferably 1 to 5, more preferably 1 to 3 hydrogen atoms, independently substituted with a corresponding number of substituents. The person skilled in the art is able to determine if the substitution is possible or impossible without paying excessive efforts by experiment or theory. For example, the combination of amino or hydroxyl group having free hydrogen and carbon atoms having unsaturated bonds (such as olefinic) may be unstable.

A “pharmaceutical composition” refers to a mixture of one or more of the compounds described in the present disclosure or physiologically/pharmaceutically acceptable salts or prodrugs thereof and other chemical components such as physiologically/pharmaceutically acceptable carriers and excipients. The purpose of a pharmaceutical composition is to facilitate administration of a compound to an organism, which is conducive to the absorption of the active ingredient and thus displaying biological activity.

“Pharmaceutically acceptable salts” refer to salts of the compounds of the disclosure, such salts being safe and effective when used in a mammal and have corresponding biological activity.

The salts can be prepared during the final isolation and purification of the compounds or separately by reacting a suitable group with a suitable alkaline or acid. Alkalines commonly employed to form pharmaceutically acceptable salts include inorganic alkalines such as sodium, potassium, lithium, calcium, magnesium, or ammonium hydroxide; organic ammonium hydroxide such as tetramethylammonium or tetraethylammonium hydroxide, as well as organic alkalines such as various organic amines, including, but not limited to, methylamine, dimethylamine, trimethylamine, triethylamine, diethylamine, ethylamine, tributylamine, pyridine, N,N-dimethylaniline, N-methylpiperidine, and N-methylmorpholine.

Acids commonly employed to form pharmaceutically acceptable salts include inorganic acids such as hydrochloric acid, hydrobromic acid, hydroiodic acid, sulfuric acid, phosphoric acid, hydrogen bisulfide, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid, acetic acid acid, and related inorganic and organic acids.

As a person skilled in the art would understand, the compounds of formula (I) or pharmaceutically acceptable salts thereof disclosed herein may exist in prodrug or solvate forms, which are all encompassed by the present disclosure.

The term “solvate,” as used herein, means a physical association of a compound of this disclosure with one or more, preferably one to three, solvent molecules, whether organic or inorganic. This physical association includes hydrogen bonding. In certain instances the solvate will be capable of isolation, for example, when one or more, preferably one to three, solvent molecules are incorporated in the crystal lattice of the crystalline solid. Exemplary solvates include, but are not limited to, hydrates, ethanolates, methanolates, and isopropanolates. Methods of solvation are generally known in the art.

“Prodrug” refers to compounds that can be transformed in vivo to yield the active parent compound under physiological conditions, such as through hydrolysis in blood.

The term “pharmaceutically acceptable,” as used herein, refers to those compounds, materials, compositions, and/or dosage forms which are, within the scope of sound medical judgment, suitable for use in contact with the tissues of patients without excessive toxicity, irritation, allergic response, or other problem or complication commensurate with a reasonable benefit/risk ratio, and are effective for their intended use.

The term “therapeutically effective amount,” as used herein, refers to the total amount of each active component that is sufficient to show a meaningful patient benefit, e.g., a sustained reduction in viral load. When applied to an individual active ingredient, administered alone, the term refers to that ingredient alone. When applied to a combination, the term refers to combined amounts of the active ingredients that result in the therapeutic effect, whether administered in combination, serially, or simultaneously.

The term “treat”, “treating”, “treatment”, or the like, refers to: (i) inhibiting the disease, disorder, or condition, i.e., arresting its development; and (ii) relieving the disease, disorder, or condition, i.e., causing regression of the disease, disorder, and/or condition. In addition, the compounds of present disclosure may be used for their prophylactic effects in preventing a disease, disorder or condition from occurring in a subject that may be predisposed to the disease, disorder, and/or condition but has not yet been diagnosed as having it.

As used herein, the singular forms “a”, “an”, and “the” include plural reference, and vice versa, unless the context clearly dictates otherwise.

The term “subject” or “patient”, as used herein, refers to a human or a mammalian animal, including but not limited to dogs, cats, horses, cows, monkeys, or the like.

When the term “about” is applied to a parameter, such as pH, concentration, temperature, or the like, it indicates that the parameter can vary by ±10%, and sometimes more preferably within ±5%. As would be understood by a person skilled in the art, when a parameter is not critical, a number is often given only for illustration purpose, instead of being limiting.

### Synthesis Methods

The compounds disclosed in the present disclosure were or can be synthesized by using the synthetic methods illustrated below:

A preparation process of preparing the compound of formula (I), or a tautomer, mesomer, racemate, enantiomer, diastereomer, or mixture thereof, or a pharmaceutically acceptable salt, solvate, or prodrug thereof, comprising a step of:

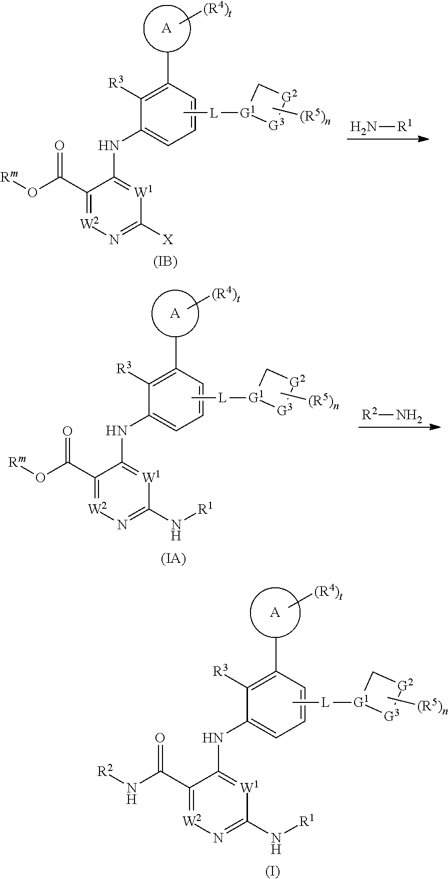

coupling a compound of formula (IB) with a compound of R¹—NH₂ under an alkaline condition with a catalyst exist to obtain the compound of formula (IA);

reacting a compound of formula (IA) with a compound of R²—NH₂ under Lewis acid (for example, MgCl₂) exist to obtain the compound of formula (I);

wherein:

R^{m} is hydrogen or alkyl; preferably C¹⁻⁶ alkyl; and

ring A, L, G¹, G², G³, W¹, W², R¹ to R⁵, n and t are each as defined in formula (I) above.

A process of preparing the compound of formula (II), or a tautomer, mesomer, racemate, enantiomer, diastereomer, or mixture thereof, or a pharmaceutically acceptable salt, solvate, or prodrug thereof, comprising a step of:

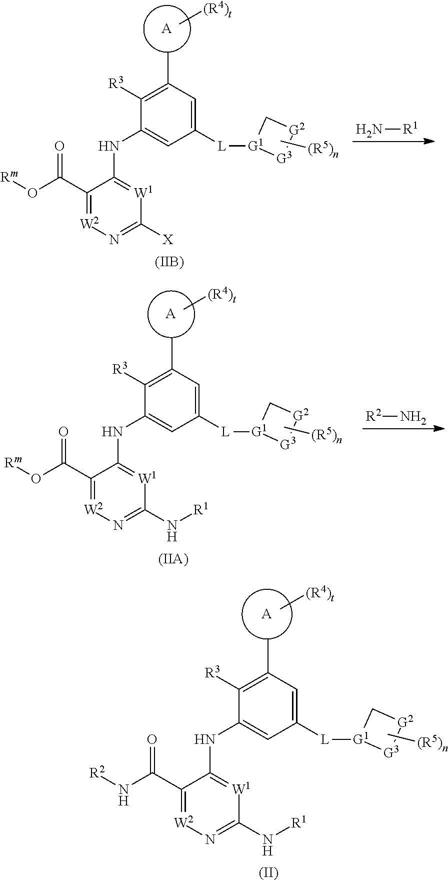

coupling a compound of formula (IIB) with a compound of R¹—NH₂ under an alkaline condition with a catalyst exist to obtain the compound of formula (IIA);

reacting a compound of formula (IIA) with a compound of R²—NH₂ under Lewis acid (for example, MgCl₂) exist to obtain the compound of formula (II);

wherein:

R^{m} is hydrogen or alkyl; preferably C₁₋₆ alkyl; and

ring A, L, G¹, G², G³, W¹, W², R¹ to R⁵, n and t are each as defined in formula (II) above.

A process of preparing the compound of formula (III), or a tautomer, mesomer, racemate, enantiomer, diastereomer, or mixture thereof, or a pharmaceutically acceptable salt, solvate, or prodrug thereof, comprising a step of:

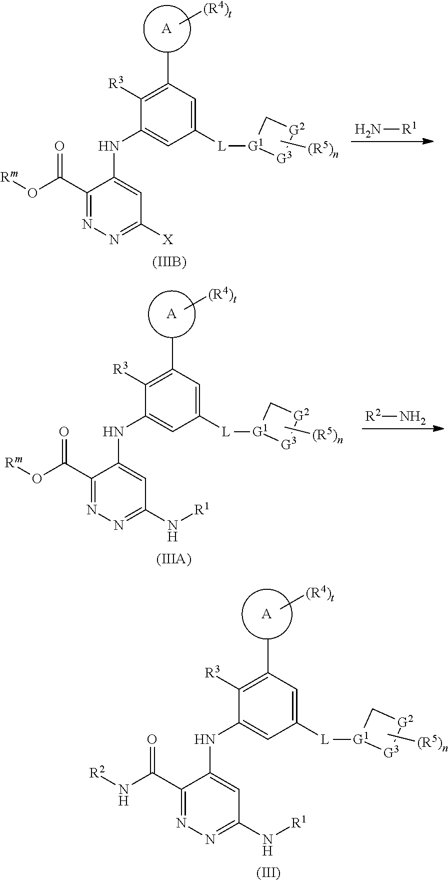

coupling a compound of formula (IIIB) with a compound of R¹—NH₂ under an alkaline condition with a catalyst exist to obtain the compound of formula (IIIA);

reacting a compound of formula (IIIA) with a compound of R²—NH₂ under Lewis acid (for example, MgCl₂) exist to obtain the compound of formula (III);

wherein:

R^{m} is hydrogen or alkyl; preferably C₁₋₆ alkyl; and

ring A, L, G¹, G², G³, R¹ to R⁵, n and t are each as defined in formula (III) above.

A process of preparing the compound of formula (IV), or a tautomer, mesomer, racemate, enantiomer, diastereomer, or mixture thereof, or a pharmaceutically acceptable salt, solvate, or prodrug thereof, comprising a step of:

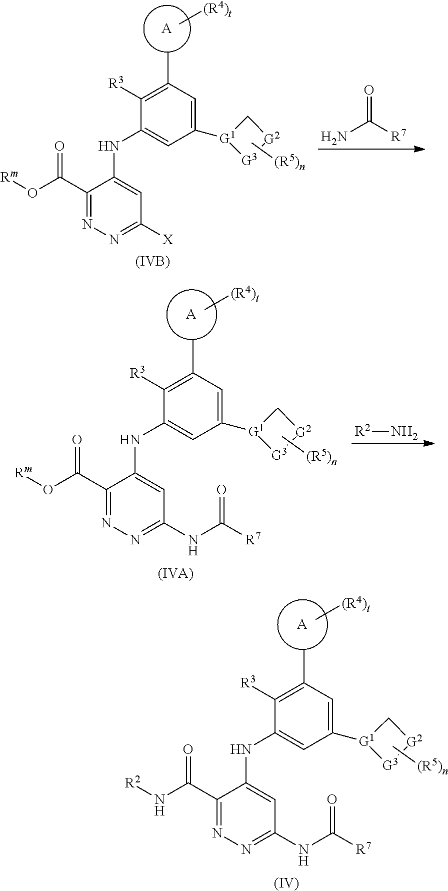

coupling a compound of formula (IVB) with a compound of R¹—NH₂ under an alkaline condition with a catalyst exist to obtain the compound of formula (IVA);

reacting a compound of formula (IVA) with a compound of R²—NH₂ under Lewis acid (for example, MgCl₂) exist to obtain the compound of formula (IV);

wherein:

R^{m} is hydrogen or alkyl; preferably C₁₋₆ alkyl;

ring A, G¹, G², G³, R² to R⁵, R⁷, n and t are each as defined in formula (IV) above.

A process of preparing the compound of formula (V), or a tautomer, mesomer, racemate, enantiomer, diastereomer, or mixture thereof, or a pharmaceutically acceptable salt, solvate, or prodrug thereof, comprising a step of:

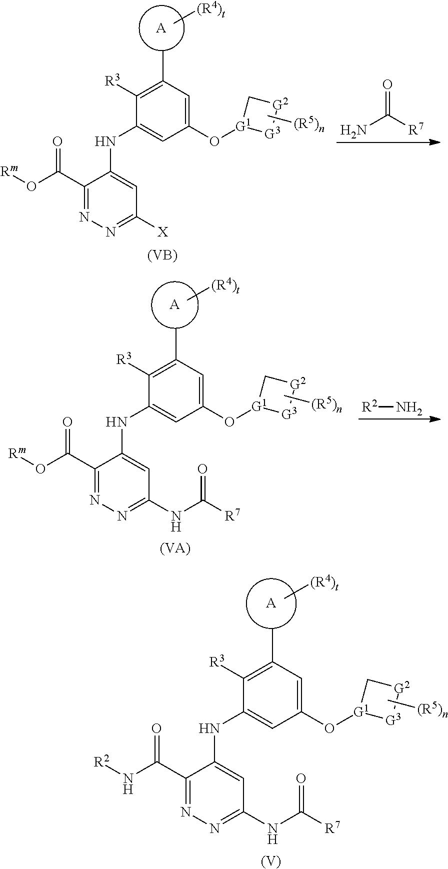

coupling a compound of formula (VB) with a compound of R¹—NH₂ under an alkaline condition with a catalyst exist to obtain the compound of formula (VA);

reacting a compound of formula (VA) with a compound of R²—NH₂ under Lewis acid (for example, MgCl₂) exist to obtain the compound of formula (V);

wherein:

R^{m} is hydrogen or alkyl; preferably C₁₋₆ alkyl;

ring A, G¹, G², G³, R² to R⁵, R⁷, n and t are each as defined in formula (V) above.

The reagent that provides an alkaline condition includes organic bases and inorganic bases. The organic bases include, but are not limited to, triethylamine, N,N-diisopropylethylamine, n-butyllithium, lithium diisopropylamide, lithium bis(trimethylsilyl)amine, potassium acetate, sodium tert-butoxide and potassium tert-butoxide. The inorganic bases include, but are not limited to, sodium hydride, potassium phosphate, sodium carbonate, potassium carbonate, potassium acetate, cesium carbonate, sodium hydroxide and lithium hydroxide; preferably cesium carbonate.

The catalyst includes, but is not limited to, tris(dibenzylideneacetone)dipalladium (Pd₂(dba)₃), XantPhos, tetrakis(triphenylphosphine)palladium, palladium dichloride, palladium acetate, [1,1′-bis(diphenylphosphino)ferrocene]dichloropalladium, 1,1′-bis(diphenylphosphino) ferrocene palladium dichloride or tris(dibenzylideneacetone)dipalladium, and preferably [1,1′-bis(diphenylphosphino)ferrocene]dichloropalladium or tetrakis(triphenylphosphine)palladium; preferably Pd₂(dba)₃/XantPhos.

The reaction is preferably in a solvent, wherein the solvent used herein includes, but is not limited to, acetic acid, methanol, ethanol, toluene, tetrahydrofuran, dichloromethane, dimethylsulfoxide, 1,4-dioxane, water, and N,N-dimethylformamide, or a mixture thereof

### EXAMPLES

The following examples serve to illustrate the invention, but the examples should not be considered as limiting. If specific conditions for the experimental method are not specified in the examples of the present disclosure, they are generally in accordance with conventional conditions or recommended conditions of the raw materials and the product manufacturer. The reagents without a specific source indicated are commercially available, conventional reagents.

The structures of compounds were determined by mass spectrometry (MS) and/or nuclear magnetic resonance (NMR). NMR shift (δ) is given in units of 10-6 (ppm).

The mass spectrum (MS) was determined using a Shimadzu LCMS-2020 liquid chromatography-mass spectrometer.

The NMR measurement was performed on a Bruker AVANCE-400 and 500 Ultrashield nuclear magnetic resonance spectrometer. The solvents were deuterated dimethylsulfoxide (DMSO-d₆), deuterated chloroform (CDCl₃) and deuterated methanol (methanol-d₄) Tetramethylsilane (TMS).

HPLC was performed using a Shimadzu OPTION BOX-L high pressure liquid phase Chromatograph (Gemini 5 um NX-C18 100×21.2 mm column).

Thin-layer chromatography (TLC) silica gel plates used were Agela Technologies T-CSF10050-M silica gel plate with size of 50 mm,

Column chromatography was commonly done using CombiFlash Rf+ Automated Flash Chromatography System (TELEDYNE ISCO) with Agela Technologies Flash Column Silica-CS prepacked columns.

Known starting materials of the present disclosure may be synthesized according to methods known in the art or may be purchased from Acros Organics, Sigma-Aldrich Chemical Company, AstaTech and other companies. Unless otherwise specified in the examples, the reactions were carried out under an argon atmosphere or a nitrogen atmosphere.

Argon or nitrogen atmosphere refers to the reaction flask connected to a volume of about 1 L argon or nitrogen balloon.

Hydrogen atmosphere refers to the reaction bottle connected to a volume of about 1 L hydrogen balloon.

Hydrogenation reaction was usually evacuated, filled with hydrogen, repeated 3 times.

The microwave reaction used a CEM Discover-S 908860 microwave reactor.

Unless otherwise specified in the examples, the reaction temperature was room temperature and was 20° C. to 30° C.

The progress of the reaction in the examples was monitored using thin layer chromatography (TLC), developing solvent for the reaction, a column chromatography eluent for purifying compound, and developing system for thin-layer chromatography include: A: dichloromethane/methanol system, B: n-hexane/ethyl acetate system, C: dichloromethane/ethyl acetate system. The volume ratio of the solvents is adjusted according to the polarity of the compound. A small amount of triethylamine and acetic acid and other alkaline or acidic reagents can be used for adjustment.

Abbreviations

CH₃NH₂ is methyl amine,

CD₃NH₂ is methyl-d₃ amine,

Cs₂CO₃ is cesium carbonate,

DIPEA is diisopropylethylamine,

DMSO is dimethyl sulfoxide,

EtOAc is ethyl acetate,

MeOH is methanol,

MgCl₂ is magnesium chloride,

MgSO₄ is magnesium sulfate,

Na₂SO₄ is sodium sulfate,

Pd₂(dba)₃ is tris(dibenzylideneacetone)dipalladium(0),

THF is tetrahydrofuran,

Xantphos is 4,5-bis(diphenylphosphino)-9,9-dimethylxanthene, and

MS is mass spectroscopy with (+) referring to the positive mode which generally gives a M+1 absorption where M=the molecular mass.

### Intermediate 1 (Int-1)

### 3-(5-bromo-2-methoxy-3-nitrophenyl)-1-methyl-1H-1,2,4-triazole

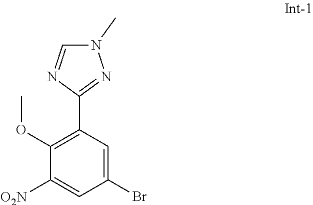 [SMILES: COc1c(-c2ncn(C)n2)cc(Br)cc1[N+](=O)[O-]]

### Synthetic Route

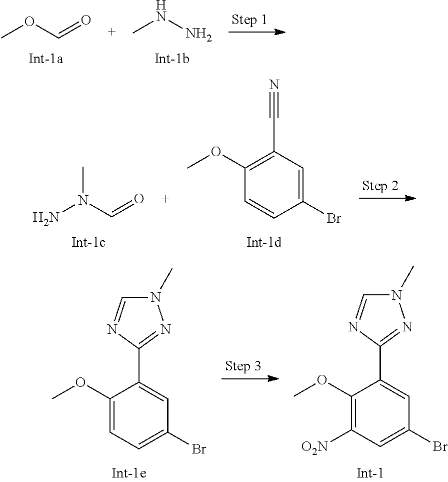

### Step 1

### N-methylformohydrazide Int-1c

To a mixture of methyl formate Int-1a (39.2 g, 653 mmol) in methanol (400 mL) under nitrogen atmosphere was added methylhydrazine Int-1b (30.0 g, 653 mmol). The resulting mixture was stirred at room temperature for 16 h, and then quenched with water (2 L) and extracted with dichloromethane (1 L×3). The combined organics were washed with brine, dried over anhydrous Na₂SO₄, and concentrated under reduced pressure to give the compound Int-1c (15 g), which was used for the next step without further purification.

### Step 2

### 3-(5-bromo-2-methoxyphenyl)-1-methyl-1H-1,2,4-triazole Int-1e

Under an argon atmosphere, potassium tert-butoxide (10.6 g, 94.3 mmol, 2.5 eq) and dry THF (100 mL) were added to an oven-dried round bottom flask containing a magnetic stirring bar. The mixture was cooled to 0° C. ˜5° C., then compound Int-1c (9.8 g, 132.04 mmol) and compound 5-bromo-2-methoxybenzonitrile Int-1d (8.0 g, 37.7 mmol) in dry tetrahydrofuran (100 mL) was added dropwise. The result mixture was stirred at 0° C. for 1.5 h, and then the mixture was poured into water (250 mL) and extracted with dichloromethane (150 mL×3). The combined organics were washed with brine, dried over anhydrous sodium sulfate and concentrated under reduced pressure. The residue was purified by chromatography over silica gel (dichloromethane:methanol=50/1) to afford 3-(5-bromo-2-methoxyphenyl)-1-methyl-1H-1,2,4-triazole Int-1e (9.0 g, 89% yield).

### Step 3

### 3-(5-bromo-2-methoxy-3-nitrophenyl)-1-methyl-1H-1,2,4-triazole Int-1

To Int-1e (10.0 g, 37.3 mmol) in dichloromethane (500 mL) was added sulfuric acid (16.5 g, 16.8 mmol), then nitric acid (3.53 g, 56 mmol) at 0° C. The result mixture was stirred at 0° C. for 30 minute, and then quenched with water (500 mL) and extracted with ethyl acetate (300 mL×3) The combined organics were washed with brine, dried over anhydrous Na₂SO₄ and concentrated under reduced pressure. The residue was purified by chromatography over silica gel (petroleum ether:ethyl acetate=6:1) to afford 3-(5-bromo-2-methoxy-3-nitrophenyl)-1-methyl-1H-1,2,4-triazole Int-1 (4.91 g, 42% yield). ¹H NMR (400 MHz, CDCl₃) δ 8.38 (s, 1H), 8.13 (s, 1H), 7.90 (m, 1H), 4.02 (s, 3H), 3.92 (s, 3H). LCMS: MS m/z (ESI): 313 [M+H]⁺.

### Intermediate 2 (Int-2)

### ethyl 6-chloro-4-((2-methoxy-5-(3-methoxyazetidin-1-yl)-3-(1-methyl-1H-1,2,4-triazol-3-yl)phenyl)amino)pyridazine-3-carboxylate

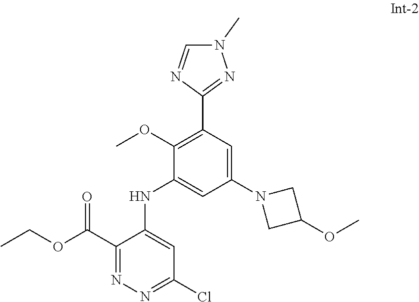 [SMILES: CCOC(=O)c1nnc(Cl)cc1Nc1cc(N2CC(OC)C2)cc(-c2ncn(C)n2)c1OC]

### Synthetic Route

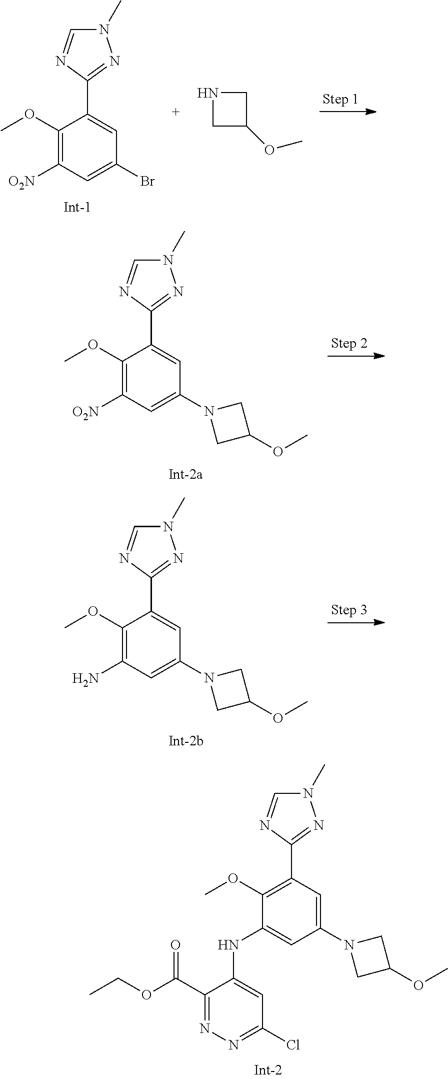

### Step 1

### 3-(2-methoxy-5-(3-methoxyazetidin-1-yl)-3-nitrophenyl)-1-methyl-1H-1,2,4-triazole Int-2a

A mixture of methyl 3-(5-bromo-2-methoxy-3-nitrophenyl)-1-methyl-1H-1,2,4-triazole Int-1 (313 mg, 1 mmol), 3-methoxyazetidine (130 mg, 1.5 mmol), Pd₂(dba)₃ (32 mg, 0.035 mmol), Xantphos (50 mg, 0.055 mmol) and Cs₂CO₃ (611 mg, 1.88 mmol) in dioxane (3 mL) under nitrogen atmosphere was sealed and heated to 100° C. for 1 hr in a Biotage initiator. After cooling down, the reaction mixture was partitioned between ethyl acetate (15 mL) and water (10 mL). The organic phase was separated and dried over Mg₂SO₄. The solid was filtered off and the solvent was evaporated. The resulting residue was purified by flash chromatography to afford 3-(2-methoxy-5-(3-methoxyazetidin-1-yl)-3-nitrophenyl)-1-methyl-1H-1,2,4-triazole Int-2a (250 mg, 78% yield). LCMS: MS m/z (ESI): 320.0 [M+H]⁺.

### Step 2

### 2-methoxy-5-(3-methoxyazetidin-1-yl)-3-(1-methyl-1H-1,2,4-triazol-3-yl)aniline Int-2b

To the MeOH (30 mL) solution of 3-(2-methoxy-5-(3-methoxyazetidin-1-yl)-3-nitrophenyl)-1-methyl-1H-1,2,4-triazole Ine-2a (250 mg, 0.78 mmol) was added Pd/C (25 mg, 10% W/W) and the mixture was hydrogenated under H2 balloon for 2 hrs. The mixture was filtered through a Celite pad. The filtrate was concentrated under vacuum to afford 2-methoxy-5-(3-methoxyazetidin-1-yl)-3-(1-methyl-1H-1,2,4-triazol-3-yl)aniline Int-2b (225 mg, 99% yield). LCMS: MS m/z (ESI): 290.0 [M+H]⁺.

### Step 3

### ethyl 6-chloro-4-((2-methoxy-5-(3-methoxyazetidin-1-yl)-3-(1-methyl-1H-1,2,4-triazol-3-yl)phenyl)amino)pyridazine-3-carboxylate Int-2

A mixture of ethyl 4,6-dichloropyridazine-3-carboxylate (205 mg, 1 mmol) and 2-methoxy-5-(3-methoxyazetidin-1-yl)-3-(1-methyl-1H-1,2,4-triazol-3-yl)aniline Int-2b (276 mg, 1 mmol) in acetonitrile (10 mL) in sealed pressure vessel under nitrogen atmosphere was added DIPEA (387 mg, 3 mmol). The resulting mixture was stirred at 130° C. for 24 hours. After cooling down, the solvent was removed under reduced pressure. The resulting residue was purified by flash chromatography (hexane/ethyl acetate) to give ethyl 6-chloro-4-((2-methoxy-5-(3-methoxyazetidin-1-yl)-3-(1-methyl-1H-1,2,4-triazol-3-yl)phenyl)amino)pyridazine-3-carboxylate Int-2 (220 mg, 47% yield). LCMS: MS m/z (ESI):474.0 [M+H]⁺.

### Example A1

### 6-(cyclopropanecarboxamido)-4-((2-methoxy-5-(3-methoxyazetidin-1-yl)-3-(1-methyl-1H-1,2,4-triazol-3-yl)phenyl)amino)-N-(methyl-d₃)pyridazine-3-carboxamide A1

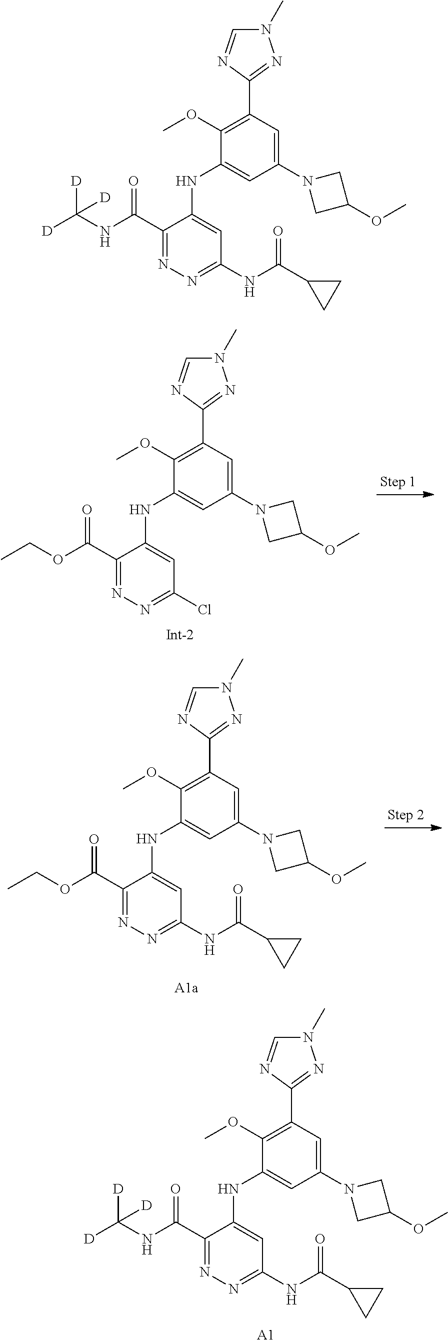

### Step 1

### Ethyl 6-(cyclopropanecarboxamido)-4-((2-methoxy-5-(3-methoxyazetidin-1-yl)-3-(1-methyl-1H-1,2,4-triazol-3-yl)phenyl)amino)pyridazine-3-carboxylate Ala

A mixture of ethyl 6-chloro-4-((2-methoxy-5-(3-methoxyazetidin-1-yl)-3-(1-methyl-1H-1,2,4-triazol-3-yl)phenyl)amino)pyridazine-3-carboxylate Int-2 (37 mg, 0.1 mmol), azetidine-1-carboxamide (14 mg, 0.14 mmol), Pd₂(dba)₃ (5 mg, 0.0055 mmol), Xantphos (5 mg, 0.0086 mmol) and Cs₂CO₃ (61 mg, 0.188 mmol) in dioxane (6 mL) under nitrogen atmosphere was sealed and heated to 85° C. for 30 min in a microwave. After cooling down, the reaction mixture was partitioned between ethyl acetate (5 mL) and water (5 mL). The organic phase was separated and dried over Mg₂SO₄. The solid was filtered off and the solvent was evaporated. The resulting residue was purified by flash chromatography to afford ethyl 6-(cyclopropanecarboxamido)-4-((2-methoxy-5-(3-methoxyazetidin-1-yl)-3-(1-methyl-1H-1,2,4-triazol-3-yl)phenyl)amino)pyridazine-3-carboxylate Ala (16 mg, 30% yield). LCMS: MS m/z (ESI): 523.0 [M+H]⁺.

### Step 2

### 6-(cyclopropanecarboxamido)-4-((2-methoxy-5-(3-methoxyazetidin-1-yl)-3-(1-methyl-1H-1,2,4-triazol-3-yl)phenyl)amino)-N-(methyl-d₃)pyridazine-3-carboxamide A1

To ethyl 6-(cyclopropanecarboxamido)-4-((2-methoxy-5-(3-methoxyazetidin-1-yl)-3-(1-methyl-1H-1,2,4-triazol-3-yl)phenyl)amino)pyridazine-3-carboxylate Ala (16 mg, 0.03 mmol) in CD₃NH₂ in THF (2N solution, 5 mL) was added magnesium chloride (3.45 mg, 0.036 mmol). The resulting mixture was stirred at room temperature for 2 h before removing solvent under reduced pressure. The resulting residue was purified by flash chromatography (hexane/EtOAc) to provide 6-(cyclopropanecarboxamido)-4-((2-methoxy-5-(3-methoxyazetidin-1-yl)-3-(1-methyl-1H-1,2,4-triazol-3-yl)phenyl)amino)-N-(methyl-d₃)pyridazine-3-carboxamide A1 (12 mg, 75% yield). ¹H NMR (400 MHz, CDCl₃) δ 10.87 (s, 1H), 9.29 (s, 1H), 8.27 (s, 1H), 8.01 (s, 1H), 7.97 (d, 1H), 6.80 (d, 1H), 6.56 (s, 1H), 4.26-4.28 (m, 1H), 4.06-4.08 (t, 2H), 3.92 (s, 3H), 3.73-3.75 (t, 2H), 3.67 (s, 3H), 3.27 (s, 3H), 1.69-1.71 (m, 1H), 0.99-1.01 (m, 2H), 0.80-0.82 (m, 2H). LCMS: MS m/z (ESI): 511.0 [M+H]⁺.

### Example A2

### 6-(cyclopropanecarboxamido)-4-((2-methoxy-5-(3-methoxyazetidin-1-yl)-3-(1-methyl-1H-1,2,4-triazol-3-yl)phenyl)amino)-N-methylpyridazine-3-carboxamide A2

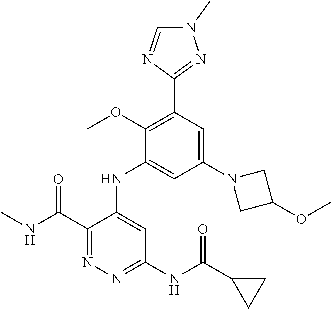

Examples A2 was prepared with the similar procedures as Example A1 by replacing CD₃NH₂ with CH₃NH₂ in step 2. ¹H NMR (400 MHz, CDCl₃): δ: 10.87 (s, 1H), 8.99 (s, 1H), 8.25 (s, 1H), 8.01 (s, 2H), 6.80 (d, 1H), 6.56 (s, 1H), 4.25-4.28 (m, 1H), 4.06-4.08 (t, 2H), 3.92 (s, 3H), 3.73-3.75 (t, 2H), 3.67 (s, 3H), 3.27 (s, 3H), 2.64-2.96 (d, 3H), 1.55-1.57 (m, 1H), 0.98-1.00 (m, 2H), 0.80-0.82 (m, 2H). LCMS: MS m/z (ESI): 508.0 [M+H]⁺.

### Example A3

### 6-(cyclopropanecarboxamido)-4-((2-methoxy-3-(1-methyl-1H-1,2,4-triazol-3-yl)-5-(2-oxoazetidin-1-yl)phenyl)amino)-N-(methyl-d₃)pyridazine-3-carboxamide A3

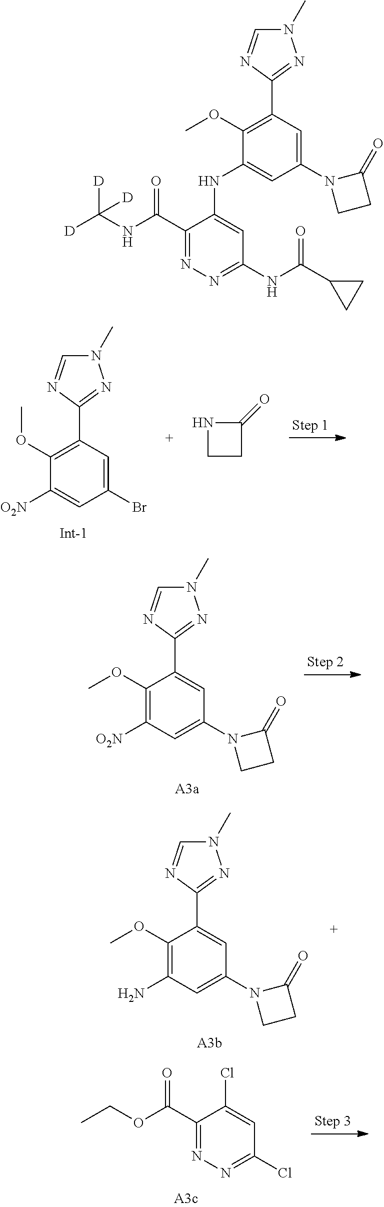

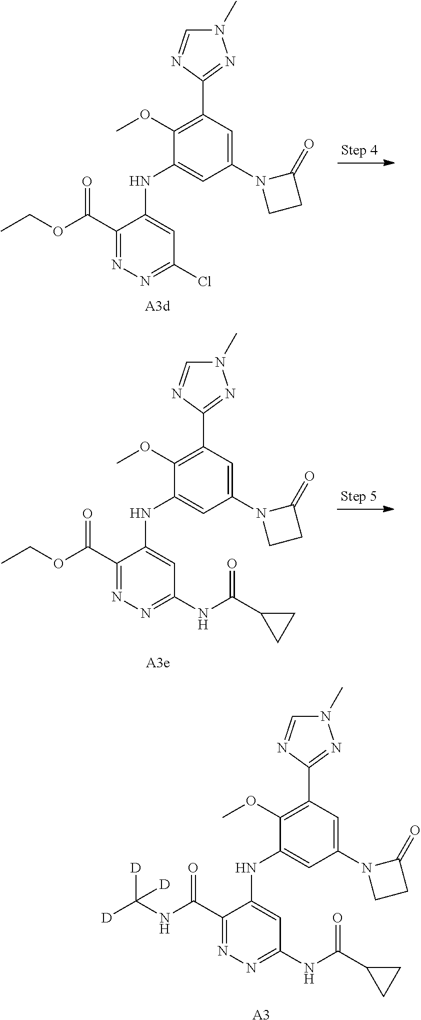 [SMILES: CCOC(=O)c1nnc(Cl)cc1Nc1cc(N2CCC2=O)cc(-c2ncn(C)n2)c1OC ; CCOC(=O)c1nnc(NC(=O)C2CC2)cc1Nc1cc(N2CCC2=O)cc(-c2ncn(C)n2)c1OC ; [2H]C([2H])([2H])NC(=O)c1nnc(NC(=O)C2CC2)cc1Nc1cc(N2CCC2=O)cc(-c2ncn(C)n2)c1OC ; "A3"]

Step 1-3 of Example A3 was prepared with the similar procedure as Int-2 by replacing 3-methoxyazetidine with 2-azetidinone in step 1.

Step 4 and 5 of Example A3 was prepared with the similar procedure as Example A1. ¹H NMR (400 MHz, CD₃OD): δ 8.44 (s, 1H), 7.67 (s, 1H), 7.56 (s, 1H), 7.41 (s, 1H), 3.92 (s, 3H), 3.64-3.67 (t, 2H), 3.61 (s, 3H), 3.04-3.06 (t, 2H), 1.77-1.79 (m. 1H), 0.97-0.99 (m, 2H), 0.91-0.93 (m, 2H). LCMS: MS m/z (ESI): 495.0 [M+H]⁺.

### Example A4

### 6-(cyclopropanecarboxamido)-4-((2-methoxy-3-(1-methyl-1H-1,2,4-triazol-3-yl)-5-(oxetan-3-yloxy)phenyl)amino)-N-(methyl-d₃)pyridazine-3-carboxamide A4

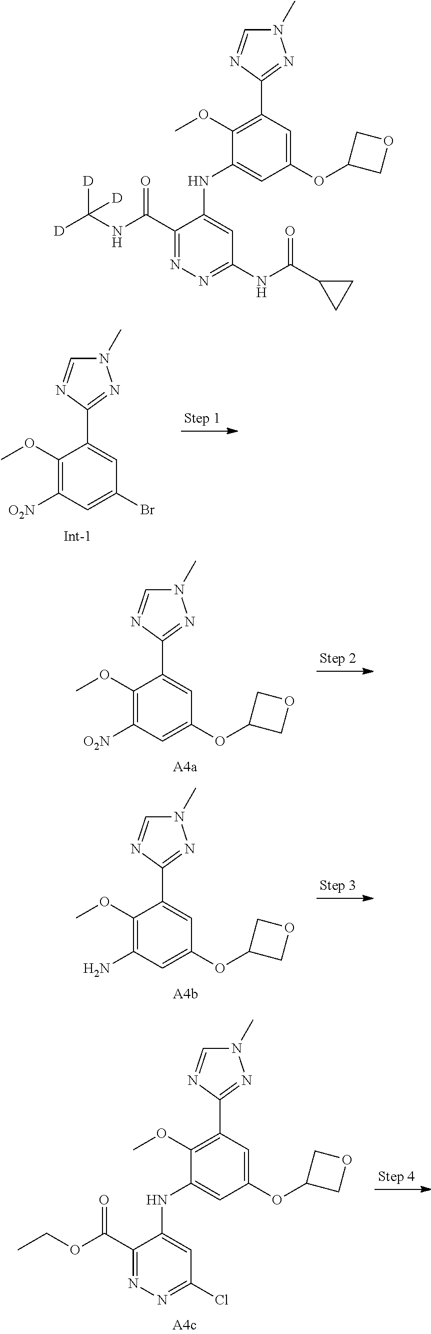 [SMILES: [2H]C([2H])([2H])NC(=O)c1nnc(NC(=O)C2CC2)cc1Nc1cc(OC2COC2)cc(-c2ncn(C)n2)c1OC ; COc1c(-c2ncn(C)n2)cc(Br)cc1[N+](=O)[O-] ; COc1c(-c2ncn(C)n2)cc(OC2COC2)cc1[N+](=O)[O-] ; COc1c(N)cc(OC2COC2)cc1-c1ncn(C)n1 ; CCOC(=O)c1nnc(Cl)cc1Nc1cc(OC2COC2)cc(-c2ncn(C)n2)c1OC]

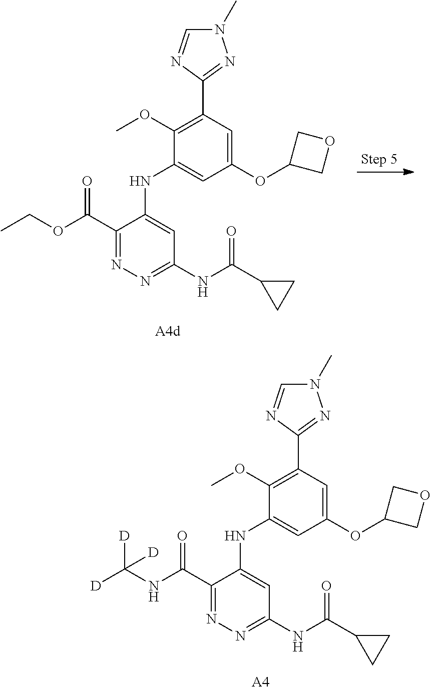 [SMILES: CCOC(=O)c1nnc(NC(=O)C2CC2)cc1Nc1cc(OC2COC2)cc(-c2ncn(C)n2)c1OC ; [2H]C([2H])([2H])NC(=O)c1nnc(NC(=O)C2CC2)cc1Nc1cc(OC2COC2)cc(-c2ncn(C)n2)c1OC ; "A4"]

### Step 1

### 3-(2-methoxy-3-nitro-5-(oxetan-3-yloxy)phenyl)-1-methyl-1H-1,2,4-triazole A4a

To the solution of compound Int-1 (2.7 g, 8.6 mmol), 3-hydroxyoxetane (639 mg, 8.6 mmol) and t-butyl Xphos (365 mg, 0.86 mmol) in toluene (10 mL) under nitrogen atmosphere was added tris(dibenzylideneacetone)dipalladium (366 mg, 0.4 mmol) and Cs₂CO₃ (5.6 g, 17.2 mmol). The reaction mixture was stirred at 80° C. for 16 h. After cooling down, the reaction mixture was filtered, and filtrate was concentrated under reduced pressure. The resulting residue was purified by column chromatography on a silica gel (petroleum ether:ethyl acetate, 1:1) to provide 3-(2-methoxy-3-nitro-5-(oxetan-3-yloxy)phenyl)-1-methyl-1H-1,2,4-triazole A4a (1.2 g, 48% yield).

### Step 2

### 2-methoxy-3-(1-methyl-1H-1,2,4-triazol-3-yl)-5-(oxetan-3-yloxy)aniline A4b

To the mixture of compound A4a (1.2 g, 3.9 mmol) in methanol (20 mL) under hydrogen atmosphere was added Pd/C (200 mg, 1.95 mmol) and sodium bicarbonate (659 mg, 7.8 mmol). The reaction mixture was stirred at room temperature for 2 h, and then filtered through Celite. The resulting filtrate was concentrated under reduced pressure, and then purified by prep-TLC (petroleum ether:ethyl acetate, 1:1) to provide 2-methoxy-3-(1-methyl-1H-1,2,4-triazol-3-yl)-5-(oxetan-3-yloxy)aniline A4b (660 mg, 61% yield). ¹H NMR (400 MHz, CD₃OD): δ 8.43 (s, 1H), 6.44-6.43 (m, 1H), 6.33-6.31 (m, 1H), 5.20 (m, 1H), 5.00-4.97 (t, J=6.6 Hz, 2H), 4.67-4.65 (m, 2H), 3.98 (s, 4H), 3.34 (s, 3H). LCMS: MS m/z (ESI): 277.2 [M+H]⁺.

Step 3 of Example A4 was prepared with the similar procedure as step 3 of Int-2 by replacing 2-methoxy-5-(3-methoxyazetidin-1-yl)-3-(1-methyl-1H-1,2,4-triazol-3-yl)aniline Int-2b with 2-methoxy-3-(1-methyl-1H-1,2,4-triazol-3-yl)-5-(oxetan-3-yloxy)aniline A4b.

Step 4 and 5 of Example A4 was prepared with the similar procedure as step 1 and 2 of Example A1. ¹H NMR (400 MHz, CD₃OD): δ 8.41 (s, 1H), 8.06 (s, 1H), 7.02 (s, 1H), 6.92 (s, 1H), 5.25-5.27 (m, 1H), 4.91-4.93 (m, 2H), 4.62-4.64 (m, 2H), 3.92 (s, 3H), 3.61 (s, 3H), 1.77-1.79 (m. 1H), 0.97-0.99 (m, 2H), 0.91-0.93 (m, 2H). LCMS: MS m/z (ESI): 498.0 [M+H]⁺.

### BIOLOGICAL ASSAYS

The present invention will be further described with reference to the following test examples, but the examples should not be considered as limiting.

### Test Example 1, KdELECT Competition Binding Assay

The KdELECT Competition Binding Assay is performed by a CRO, Eurofins DiscoverX Corporation following the established standard protocol (DiscoverX, San Diego, Calif.). Briefly, Kinase-tagged T7 phage strains were prepared in an E. coli host derived from the BL21 strain. E. coli were grown to log-phase and infected with T7 phage and incubated with shaking at 32° C. until lysis. The lysates were centrifuged and filtered to remove cell debris. The remaining kinases were produced in HEK-293 cells and subsequently tagged with DNA for qPCR detection. Streptavidin-coated magnetic beads were treated with biotinylated small molecule ligands for 30 minutes at room temperature to generate affinity resins for kinase assays. The liganded beads were blocked with excess biotin and washed with blocking buffer (SeaBlock (Pierce), 1% BSA, 0.05% Tween 20, 1 mM DTT) to remove unbound ligand and to reduce nonspecific binding. Binding reactions were assembled by combining kinases, liganded affinity beads, and test compounds in 1× binding buffer (20% SeaBlock, 0.17×PBS, 0.05% Tween 20, 6 mM DTT). Test compounds were prepared as 111-fold stocks in 100% DMSO. Kds were determined using an 11-point 3-fold compound dilution series with three DMSO control points. All compounds for Kd measurements are distributed by acoustic transfer (non-contact dispensing) in 100% DMSO. The compounds were then diluted directly into the assays such that the final concentration of DMSO was 0.9%. All reactions performed in polypropylene 384-well plate. Each was a final volume of 0.02 mL. The assay plates were incubated at room temperature with shaking for 1 hour and the affinity beads were washed with wash buffer (1×PBS, 0.05% Tween 20). The beads were then re-suspended in elution buffer (1×PBS, 0.05% Tween 20, 0.5 μM non-biotinylated affinity ligand) and incubated at room temperature with shaking for 30 minutes. The kinase concentration in the eluates was measured by quantitative PCR.

Compound Handling: An 11-point 3-fold serial dilution of each test compound was prepared in 100% DMSO at 100-fold final test concentration and subsequently diluted to 1-fold in the assay (final DMSO concentration=1%). Most Kds were determined using a compound top concentration=30,000 nM. If the initial Kd determined was <0.5 nM (the lowest concentration tested), the measurement was repeated with a serial dilution starting at a lower top concentration. A Kd value reported as 40,000 nM indicates that the Kd was determined to be >30,000 nM.

KdELECT Data

| Example No. | KdELECT (Kd, nM) |
|---|---|
| A1 | 0.0035 |
| A2 | 0.004 |
| A3 | 0.0071 |
| A4 | 0.037 |

Conclusion: The compounds of the present disclosure have a significant inhibition effect on the Tyk2 JH2 Pseudokinase and ligand binding, thus revealing high affinity of compounds to Tyk2 JH2.

### Test Example 2, IL-23_Kit225 T Cell Assay

Kit225 cells (licensed from Professor Toshiyuki Hori, Ritsumeikan University, Japan) were seeded in 384-well plate at a density of 1×10⁵ cells/well in 4 μL Hank's Balanced Salt Solution (HBSS, Gibco), and incubated for 2 hours in a humidified, 5% CO₂ cell culture incubator at 37° C. The cells were treated with serial diluted compounds for 1 hour and stimulated with human recombinant IL-23 (R&D Systems) for 20 minutes. The treated cells were then lysed and cellular phosphorylated-STAT3 levels were measured by AlphaLISA (PerkinElmer) according to the manufacture's instructions. Inhibition data were calculated by comparison to vehicle control wells for 0% inhibition and non-stimulated control wells for 100% inhibition. Dose response curves were then generated to determine the concentration required to suppress 50% of cellular response (IC₅₀) as derived by non-linear regression analysis using GraphPad Prism.

Kit225 T Cell Inhibition Data

| Example No. | IL-23 Kit225 (IC₅₀, μM) |
|---|---|
| A1 | 0.057 |
| A2 | 0.088 |
| A3 | 0.0097 |
| A4 | 0.13 |

Conclusion: The compounds of the present disclosure have a significant inhibition effect on the Tyk2-mediated, IL23-induced STAT3 phosphorylation in human Kit225 T cell line.

### Test Example 3 IFNα_Kit225 T Cell Assay

Kit225 cells were seeded in 384-well plate at a density of 0.5×10⁵ cells/well in 4 μL HBSS, and incubated for 2 hours in a humidified, 5% CO₂ cell culture incubator at 37° C. The cells were treated with serial diluted compounds for 1 hour and stimulated with human recombinant IFNα (Biolegend) for 20 minutes. The treated cells were then lysed and cellular phosphorylated-STAT1 levels were measured by AlphaLISA (PerkinElmer) according to the manufacturer's instructions. Inhibition data were calculated by comparison to vehicle control wells for 0% inhibition and non-stimulated control wells for 100% inhibition. Dose response curves were then generated to determine the concentration required to suppress 50% of cellular response (IC₅₀) as derived by non-linear regression analysis using GraphPad Prism.

IFNα_Kit225 T Cell Inhibition Data

| Example No. | IFN-α Kit225 (IC₅₀, μM) |
|---|---|
| A1 | 0.043 |
| A2 | 0.050 |
| A3 | 0.0075 |
| A4 | 0.41 |

Conclusion: The compounds of the present disclosure have a significant inhibition effect on the Tyk2-mediated, IFNα-induced STAT1 phosphorylation in human Kit225 T cell line.

The foregoing embodiments and examples are provided for illustration only and are not intended to limit the scope of the invention. Various changes and modifications to the disclosed embodiments will be apparent to those skilled in the art based on the present disclosure, and such changes and modifications may be made without departure from the spirit and scope of the present invention. All literature cited are incorporated herein by reference in their entireties without admission of them as prior art.

What is claimed is:

1. A compound of formula (I), or a tautomer, mesomer, racemate, enantiomer, diastereomer, or mixture thereof, or a pharmaceutically acceptable salt, solvate, or prodrug thereof: 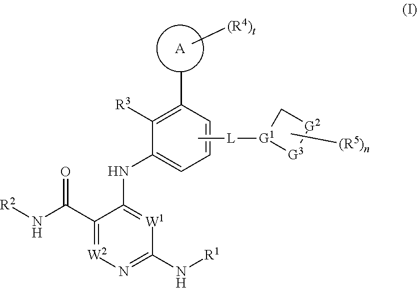
wherein:
G¹ is N atom or C atom;
G² and G³ are identical or different, and each is independently selected from the group consisting of C atom, O atom, N atom and S atom, provided that at least one of G¹, G² and G³ is a heteroatom;
W¹ and W² are identical or different, and each is independently N atom or CR⁶;
L is selected from the group consisting of a bond, O atom and alkylene, wherein the alkylene is optionally substituted with one or more groups independently selected from the group consisting of halogen, alkyl, alkoxy, hydroxyl, hydroxyalkyl, cyano, amino, nitro, cycloalkyl, heterocyclyl, aryl and heteroaryl;
ring A is heteroaryl;
R¹ is selected from the group consisting of hydrogen, alkyl, deuterated alkyl, haloalkyl, hydroxyalkyl, alkenyl, alkynyl and —C(O)R⁷;
R² is selected from the group consisting of hydrogen, halogen, alkyl, deuterated alkyl, alkenyl, alkynyl, alkoxy, haloalkyl, haloalkoxy, hydroxyl, hydroxyalkyl, cyano, amino, nitro, cycloalkyl and heterocyclyl;
R³ is selected from the group consisting of hydrogen, halogen, alkyl, deuterated alkyl, alkenyl, alkynyl, alkoxy, haloalkyl, haloalkoxy, hydroxyl, hydroxyalkyl, cyano, amino nitro, cycloalkyl and heterocyclyl;
R⁴ at each occurrence is independently selected from the group consisting of hydrogen, halogen, alkyl, deuterated alkyl, alkenyl, alkynyl, alkoxy, haloalkyl, haloalkoxy, hydroxyl, hydroxyalkyl, cyano, amino, nitro, cycloalkyl, heterocyclyl, aryl and heteroaryl;
R⁵ at each occurrence is independently selected from the group consisting of hydrogen, halogen, alkyl, oxo, alkenyl, alkynyl, alkoxy, haloalkyl, haloalkoxy, hydroxyl, hydroxyalkyl, cyano, amino, nitro, cycloalkyl, heterocyclyl, aryl and heteroaryl;
R⁶ is selected from the group consisting of hydrogen, halogen, alkyl, alkenyl, alkynyl, deuterated alkyl, alkoxy, haloalkyl, haloalkoxy, hydroxyl, hydroxyalkyl, cyano, amino, nitro, cycloalkyl, heterocyclyl, aryl and heteroaryl;
R⁷ is selected from the group consisting of alkyl, alkenyl, alkynyl, haloalkyl, hydroxyalkyl, cycloalkyl and heterocyclyl;
n is 0, 1, 2, 3 or 4; and
t is 0, 1, 2 or 3.

2. The compound of formula (I), or a tautomer, mesomer, racemate, enantiomer, diastereomer, or mixture thereof, or a pharmaceutically acceptable salt, solvate, or prodrug thereof according to claim 1, being a compound of formula (II), or a tautomer, mesomer, racemate, enantiomer, diastereomer, or mixture thereof, or a pharmaceutically acceptable salt, solvate, or prodrug thereof: 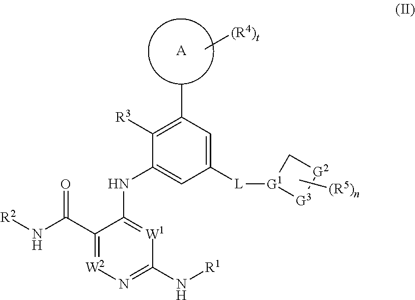
wherein:
ring A, L, G¹, G², G³, W¹, W², R¹ to R⁵, n and t are each as defined in claim 1.

3. The compound of formula (I), or a tautomer, mesomer, racemate, enantiomer, diastereomer, or mixture thereof, or a pharmaceutically acceptable salt, solvate, or prodrug thereof according to claim 1, wherein W¹ is CR⁶, and W² is N atom; and R⁶ is as defined in claim 1.

4. The compound of formula (I), or a tautomer, mesomer, racemate, enantiomer, diastereomer, or mixture thereof, or a pharmaceutically acceptable salt, solvate, or prodrug thereof according to claim 1, being a compound of formula (III), or a tautomer, mesomer, racemate, enantiomer, diastereomer, or mixture thereof, or a pharmaceutically acceptable salt, solvate, or prodrug thereof: 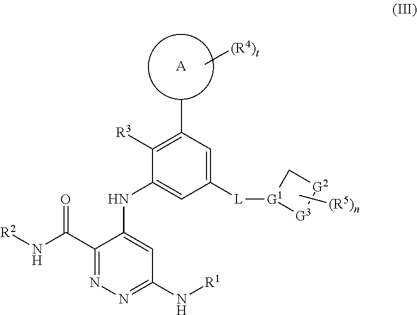
wherein:
ring A, L, G¹, G², G³, R¹ to R⁵, n and t are each as defined in claim 1.

5. The compound of formula (I), or a tautomer, mesomer, racemate, enantiomer, diastereomer, or mixture thereof, or a pharmaceutically acceptable salt, solvate, or prodrug thereof according to claim 1, wherein R¹ is —C(O)R⁷, and R⁷ is as defined in claim 1.

6. The compound of formula (I), or a tautomer, mesomer, racemate, enantiomer, diastereomer, or mixture thereof, or a pharmaceutically acceptable salt, solvate, or prodrug thereof according to claim 1, wherein L is a bond or O atom.

7. The compound of formula (I), or a tautomer, mesomer, racemate, enantiomer, diastereomer, or mixture thereof, or a pharmaceutically acceptable salt, solvate, or prodrug thereof according to claim 1, being a compound of formula (IV) or formula (V), or a tautomer, mesomer, racemate, enantiomer, diastereomer, or mixture thereof, or a pharmaceutically acceptable salt, solvate, or prodrug thereof: 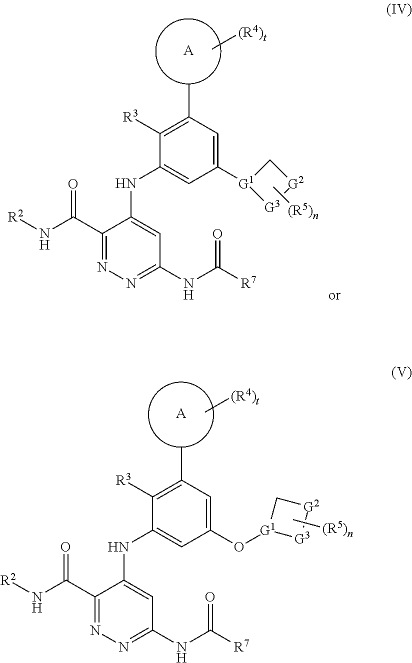
wherein:
ring A, G¹, G², G³, R² to R⁵, R⁷, n and t are each as defined in claim 1.

8. The compound of formula (I), or a tautomer, mesomer, racemate, enantiomer, diastereomer, or mixture thereof, or a pharmaceutically acceptable salt, solvate, or prodrug thereof according to claim 1, wherein 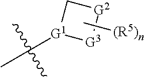
is selected from the group consisting of 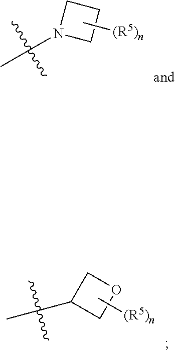
and R⁵ is as defined in claim 1.

9. The compound of formula (I), or a tautomer, mesomer, racemate, enantiomer, diastereomer, or mixture thereof, or a pharmaceutically acceptable salt, solvate, or prodrug thereof according to claim 1, wherein R⁵ is each identical or different, and each is independently selected from the group consisting of hydrogen, halogen, C₁₋₆alkyl, oxo, C₁₋₆alkoxy, haloC₁₋₆alkyl, haloC₁₋₆alkoxy, hydroxyl and hydroxy C₁₋₆alkyl.

10. The compound of formula (I), or a tautomer, mesomer, racemate, enantiomer, diastereomer, or mixture thereof, or a pharmaceutically acceptable salt, solvate, or prodrug thereof according to claim 1, wherein 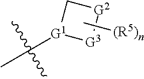
is selected from the group consisting of

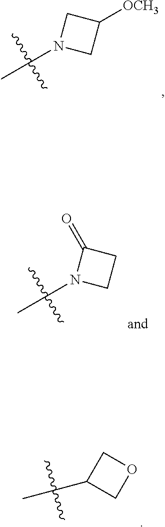 [SMILES: *N1CC(OC)C1 ; "," ; *N1CCC1=O ; *C1COC1 ; "."]

11. The compound of formula (I), or a tautomer, mesomer, racemate, enantiomer, diastereomer, or mixture thereof, or a pharmaceutically acceptable salt, solvate, or prodrug thereof according to claim 1, wherein ring A is 5-member heteroaryl; and preferably triazolyl.

12. The compound of formula (I), or a tautomer, mesomer, racemate, enantiomer, diastereomer, or mixture thereof, or a pharmaceutically acceptable salt, solvate, or prodrug thereof according to claim 1, wherein R² is C₁₋₆alkyl or deuterated C₁₋₆alkyl.

13. The compound of formula (I), or a tautomer, mesomer, racemate, enantiomer, diastereomer, or mixture thereof, or a pharmaceutically acceptable salt, solvate, or prodrug thereof according to claim 1, wherein R³ is C₁₋₆alkoxy.

14. The compound of formula (I), or a tautomer, mesomer, racemate, enantiomer, diastereomer, or mixture thereof, or a pharmaceutically acceptable salt, solvate, or prodrug thereof according to claim 1, wherein R⁴ is each identical or different, and each is independently selected from the group consisting of hydrogen and C₁₋₆alkyl.

15. The compound of formula (I), or a tautomer, mesomer, racemate, enantiomer, diastereomer, or mixture thereof, or a pharmaceutically acceptable salt, solvate, or prodrug thereof according to claim 1, wherein R⁷ is C₃₋₆cycloalkyl.

16. The compound of formula (I), or a tautomer, mesomer, racemate, enantiomer, diastereomer, or mixture thereof, or a pharmaceutically acceptable salt, solvate, or prodrug thereof according to claim 1, wherein the compound is selected from the group consisting of:

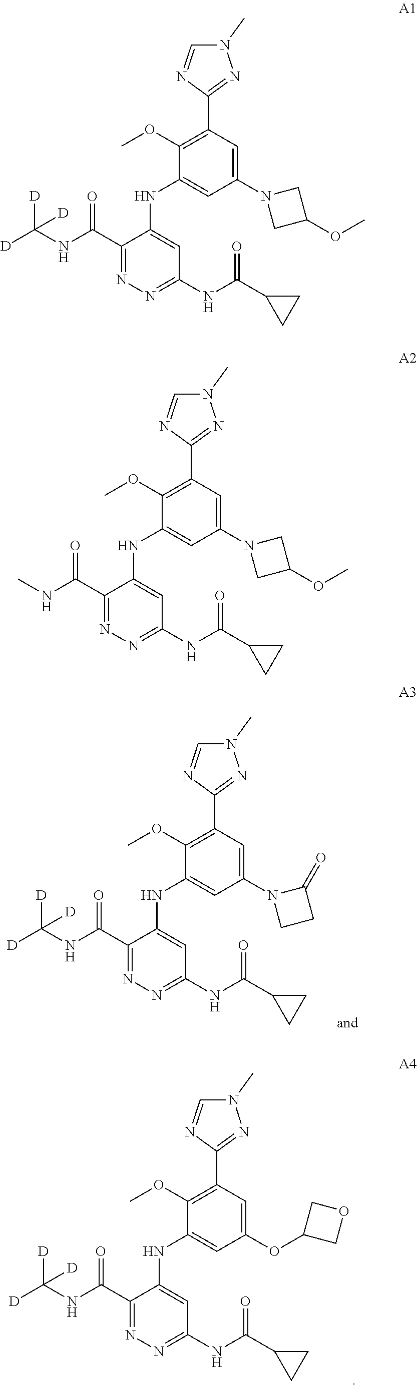

17. A compound of formula (IA), or a tautomer, mesomer, racemate, enantiomer, diastereomer, or mixture thereof, or a pharmaceutically acceptable salt, solvate, or prodrug thereof: 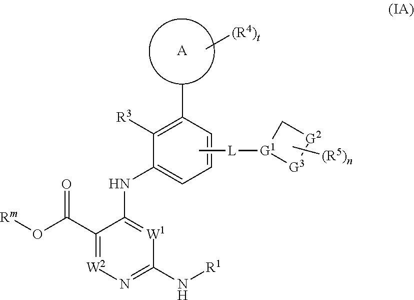
Wherein:
R^{m} is hydrogen or alkyl; preferably C₁₋₆ alkyl;
ring A, L, G¹, G², G³, W¹, W², R³ to R⁵, n and t are each as defined in claim 1.

18. The compound of formula (IA), or a tautomer, mesomer, racemate, enantiomer, diastereomer, or mixture thereof, or a pharmaceutically acceptable salt, solvate, or prodrug thereof according to claim 17, wherein the compound is selected from the group consisting of:

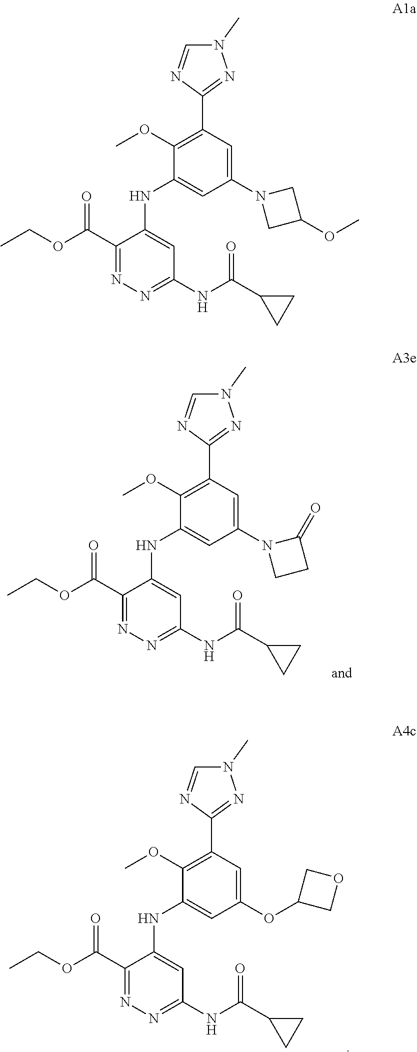

19. A process of preparing the compound of formula (I) according to claim 1, or a tautomer, mesomer, racemate, enantiomer, diastereomer, or mixture thereof, or a pharmaceutically acceptable salt, solvate, or prodrug thereof, comprising a step of: 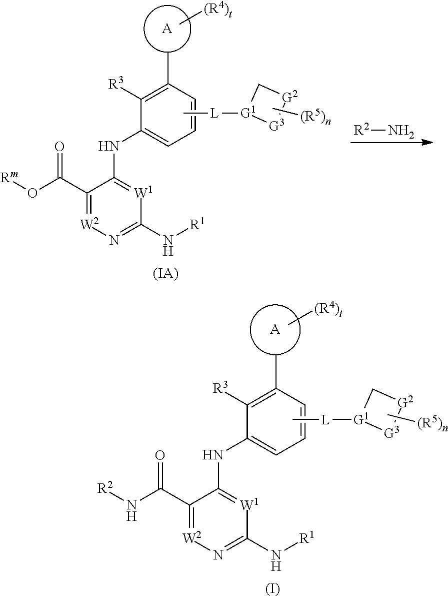
reacting a compound of formula (IA) with a compound of R²—NH₂ to obtain the compound of formula (I);
wherein:
R^{m} is hydrogen or alkyl; preferably C₁₋₆ alkyl;
ring A, L, G¹, G², G³, W¹, W², R¹ to R⁵, n and t are each as defined in claim 1.

20. A pharmaceutical composition comprising a therapeutically effective amount of the compound of formula (I), or a tautomer, mesomer, racemate, enantiomer, diastereomer, or mixture thereof, or a pharmaceutically acceptable salt, solvate, or prodrug thereof according to claim 1, and a pharmaceutically acceptable carrier.

21. A method of treating an Tyk2-mediated disorder, disease or condition, comprising a step of administering to a subject in need thereof a therapeutically effective amount of the compound of formula (I), or a tautomer, mesomer, racemate, enantiomer, diastereomer, or mixture thereof, or a pharmaceutically acceptable salt, solvate, or prodrug thereof according to claim 1, or a pharmaceutical composition thereof.

22. A method of treating proliferative, metabolic, allergic, autoimmune and inflammatory diseases, comprising a step of administering to a subject in need thereof a therapeutically effective amount of the compound of formula (I), or a tautomer, mesomer, racemate, enantiomer, diastereomer, or mixture thereof, or a pharmaceutically acceptable salt, solvate, or prodrug thereof according to claim 1, or a pharmaceutical composition thereof.

23. A method of treating autoimmune and inflammatory diseases, comprising a step of administering to a subject in need thereof a therapeutically effective amount of the compound of formula (I), or a tautomer, mesomer, racemate, enantiomer, diastereomer, or mixture thereof, or a pharmaceutically acceptable salt, solvate, or prodrug thereof according to claim 1, or a pharmaceutical composition thereof.

24. The method according to claim 23, wherein the autoimmune and inflammatory diseases is selected from arthritis, rheumatoid arthritis, multiple sclerosis, systemic lupus erythematosus, lupus nephritis, cutaneous lupus, inflammatory bowel disease, psoriasis, psoriatic arthritis, Crohn's disease, Sjögren's syndrome, systemic scleroderma, ulcerative colitis, Graves' disease, discoid lupus erythematosus, adult onset stills, systemic onset juvenile idiopathic arthritis, gout, gouty arthritis, type I diabetes, insulin dependent diabetes mellitus, sepsis, septic shock, Shigellosis, pancreatitis, glomerulonephritis, autoimmune gastritis, diabetes, autoimmune hemolytic anemia, autoimmune neutropenia, thrombocytopenia, atopic dermatitis, myasthenia gravis, ankylosing spondylitis, pemphigus vulgaris, Goodpasture's disease, antiphospholipid syndrome, idiopathic thrombocytopenia, ANCA-associated vasculitis, pemphigus, Kawasaki disease, Chronic Inflammatory Demyelinating Polyneuropathy (CIDP), dermatomyositis, polymyositis, uveitis, Guillain-Barre syndrome, autoimmune pulmonary inflammation, autoimmune thyroiditis, autoimmune inflammatory eye disease and chronic demyelinating polyneuropathy.

25. A method of treating cancer, comprising a step of administering to a subject in need thereof a therapeutically effective amount of the compound of formula (I), or a tautomer, mesomer, racemate, enantiomer, diastereomer, or mixture thereof, or a pharmaceutically acceptable salt, solvate, or prodrug thereof according to claim 1, or a pharmaceutical composition thereof.
